(19) United States
(12) Patent Application Publication
(10) Pub. No.: US 2013/0178751 A1
(43) Pub. Date: Jul. 11, 2013

(54) IMPLANTABLE MEDICAL DEVICE FOR MEASURING PRESSURE VIA AN L-C RESONANT CIRCUIT

(71) Applicant: Xiaoyi Min, Camarillo, CA (US)

(72) Inventor: Xiaoyi Min, Camarillo, CA (US)

(73) Assignee: PACESETTER, INC., Sylmar, CA (US)

(21) Appl. No.: 13/778,007

(22) Filed: Feb. 26, 2013

Related U.S. Application Data
(63) Continuation-in-part of application No. 13/333,734, filed on Dec. 21, 2011.

(51) Int. Cl. A61B 5/0215

(52) U.S. Cl. CPC A61B 5/0215
USPC 600/486

(57) ABSTRACT

An implantable medical device controls the excitation of and processes signals received from passive pressure sensor components of an implantable lead. The passive pressure sensor components include an inductor-capacitor (L-C) resonant circuit that has a resonant frequency that corresponds in some aspects to the pressure external to the implantable lead. The capacitive circuit portion of the resonant circuit may be flexible such that changes in pressure at the capacitive circuit cause changes in the capacitance of the capacitive circuit. Thus, changes in pressure at the pressure sensor are reflected by changes in the resonant frequency of the excited resonant circuit. The L-C resonant circuit is excited by a signal coupled to the L-C resonant circuit by the implantable medical device. In some embodiments, the implantable medical device receives such an excitation signal from an external device. In some embodiments, the implantable medical device generates the excitation signal.

### CROSS REFERENCE TO RELATED APPLICATIONS

This application is a continuation-in-part of U.S. patent application Ser. No. 13/333,734, filed Dec. 21, 2011, titled “Passive Pressure Sensor for Implantable Lead.”

### TECHNICAL FIELD

This application relates generally to implantable medical devices and more specifically, but not exclusively, to controlling pressure measurements.

### BACKGROUND

When a person's heart does not function normally due to, for example, a genetic or acquired condition, various treatments may be prescribed to correct or compensate for the condition. For example, pharmaceutical therapy may be prescribed for a patient or a pacemaker or similar device may be implanted in the patient to improve the function of the patient's heart.

In conjunction with such therapy, it may be desirable to detect conditions in or apply therapy to one or more chambers of the heart. For example, the health of many patients who have had some form of heart failure (e.g., a heart attack) may deteriorate over time due to progressive failure of the heart.

Heart failure is a debilitating disease in which abnormal function of a patient's heart leads to inadequate blood flow to the patient's body. While a heart failure patient may not suffer debilitating symptoms immediately, with few exceptions, the disease is relentlessly progressive. Moreover, as heart failure progresses, it may become increasingly difficult to manage.

Despite current drug and device therapies, the rate of heart failure hospitalization remains high. Consequently, significant hospitalizations costs are incurred annually for heart failure patients.

Cardiac pressure monitoring has been suggested as a means for tracking heart failure progression in a patient. For example, pulmonary artery pressure has been proposed as a predictor for heart failure progression. In addition, a rise in left atrial pressure has been proposed as a potential indicator of left ventricular failure.

Consequently, it has been proposed to implant pressure sensors that will monitor cardiac pressure in various chambers. For example, it has proposed to incorporate active pressure sensors on implantable leads to measure ventricular pressure or atrial pressure. In addition, it has been proposed to place a dedicated pressure sensor in a branch of the pulmonary artery for heart failure monitoring. However, these types of sensors are generally quite complicated and have a relatively high cost. In addition, there may be risks associated a dedicated implant procedure used for dedicated sensors.

Accordingly, a need exists for more effective techniques for monitoring pressure so that appropriate treatment may be readily prescribed for patients, thereby lowering the hospitalization rate for the patients.

### SUMMARY

A summary of several sample aspects of the disclosure follows. This summary is provided for the convenience of the reader to provide a basic understanding of such aspects and does not wholly define the breadth of the disclosure. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later. For convenience, the term some aspects may be used herein to refer to a single aspect or multiple aspects of the disclosure. Similarly, the term some embodiments may be used herein to refer to a single embodiment or multiple embodiments.

The disclosure relates in some aspects to control mechanisms for passive pressure sensor components that are incorporated into an implantable lead. For example, control mechanisms in an implantable medical device (e.g., a pacemaker, a cardioverter defibrillator, etc.) may control the excitation of and process signals received from passive pressure sensor components of a pacing lead and/or sensing lead, a high voltage lead, or some other type of lead.

The passive pressure sensor components include an inductor-capacitor (L-C) resonant circuit that has a resonant frequency that corresponds in some aspects to the pressure external to the implantable lead. For example, the capacitive circuit portion of the resonant circuit may be flexible and mechanically coupled to an exterior portion of the implantable lead. Consequently, changes in pressure at the pressure sensor (e.g., changes in cardiac pressure at a lead implanted in a patient's heart) cause a change in the physical characteristics of the capacitive circuit and thereby change the capacitance of the capacitive circuit. Thus, changes in pressure at the pressure sensor are reflected by changes in the resonant frequency of the excited resonant circuit.

The L-C resonant circuit is excited by a signal coupled to the L-C resonant circuit by the implantable medical device. For example, such an excitation signal may be carried by a pair of lead conductors that run from the L-C resonant circuit to a lead connector that interfaces with a corresponding connector of the implantable medical device. In some aspects, this excitation signal has a frequency that is substantially equal to (i.e., equal to or approximately equal to) the nominal resonant frequency of the L-C resonant circuit.

In some embodiments, the implantable medical device receives such an excitation signal from an external device. For example, the implantable medical device may include an antenna configured to receive a radio frequency (RF) signal from an external device whereby, at appropriate times, the implantable medical device couples the received RF signal to the L-C resonant circuit of the lead (e.g., via the above lead conductors).

Such a configuration may more efficiently couple an RF signal to an L-C resonant circuit of a lead as compared to systems where an L-C resonant circuit-based pressure sensor of a lead directly receives an RF signal from an external device. For example, an implantable medical device incorporating circuitry based on the teachings herein may be implanted subcutaneously (e.g., near a patient's chest) while a cardiac lead is implanted within the heart. Thus, the implantable medical device will be able to receive an externally generated signal with less loss as compared to a sensor implanted within the heart since a signal received by the implantable medical device will have passed through less tissue as compared to a signal received by the above lead-based sensor.

In some embodiments, the implantable medical device generates the excitation signal provided to the L-C resonant circuit. For example, the implantable medical device may include a signal generator configured to generate the excitation signal whereby, at appropriate times, the implantable medical device couples the generated signal to the L-C resonant circuit of the lead (e.g., via the above lead conductors).

The implantable medical device includes circuitry for detecting the current resonant frequency of the L-C resonant circuit. For example, after coupling an excitation signal to the L-C resonant circuit for a period of time, the implantable medical device may decouple the excitation signal from the L-C resonant circuit and then sense the oscillating signal generated by the excited L-C resonant circuit. As the actual resonant frequency of the L-C resonant circuit will vary depending on the pressure at the L-C resonant circuit, this pressure value may be calculated by determining the frequency of the oscillating signal generated by the excited L-C resonant circuit and received by the implantable medical device. Data generated based on this received signal may thus be uploaded from the implantable medical device to an external monitoring system. In this way, pressure measurement information may be provided to a physician, a clinician, the patient, or some other person or entity.

Accordingly, a passive pressure sensor as taught herein may be effectively employed to monitor and, therefore, treat heart failure (e.g., by monitoring changes in blood pressure that are indicative of heart failure). For example, when incorporated with an RV lead, the passive pressure sensor may be used to measure RV pressure, dP/dt, and estimated pulmonary artery pressure. To this end, the passive pressure sensor may be located at various locations along the implantable lead, whereby the implantable lead is oriented upon implant to place the passive pressure sensor at a desired location within the heart.

There are several potential advantages over existing systems provided by a lead-based passive pressure sensor as taught herein. No significant added surgical procedures or implant time is needed since the pressure sensor may be implanted with or in conjunction with implant of the lead. There is less clinical risk since the pressure sensor may be fully integrated into a standard lead. There is less clinical risk since the pressure sensor need not be implanted in the pulmonary artery or across the intra-atrial septum. There is lower cost due to the use of low complexity circuits. Portability may be improved since a more efficient telemetry design that is integrated with the whole system of a programmer, a pacer/ICD/CRT, and a telemetry system may be employed.

### BRIEF DESCRIPTION OF THE DRAWINGS

These and other aspects of the disclosure will be more fully understood when considered with respect to the following detailed description, the appended claims, and the accompanying drawings, wherein:

FIG. 1 is a simplified diagram of a system including an implantable medical device and an implantable lead incorporating passive pressure components;

FIG. 2 is a simplified diagram of an implantable medical device configured to selectively couple received RF signals to an implantable lead, where the implantable medical device incorporates components for measuring pressure via an L-C resonant circuit of the implantable lead;

FIGS. 3 and 4 are a flowchart of an embodiment of operations performed to conduct pressure measurements;

FIG. 5 is a simplified diagram of an implantable medical device configured to selectively couple generated signals to an implantable lead, where the implantable medical device incorporates components for measuring pressure via an L-C resonant circuit of the implantable lead;

FIG. 6 is a flowchart of another embodiment of operations performed to conduct pressure measurements;

FIG. 7 is a simplified diagram of an embodiment of a medical system illustrating communication between an implantable medical device and external devices;

FIG. 8 is a simplified diagram of another embodiment of a medical system illustrating communication between an implantable medical device and an external device;

FIG. 9 is a simplified diagram of a distal section of an implantable lead incorporating a passive pressure component;

FIG. 10 is a simplified diagram of view A of the implantable lead of FIG. 9;

FIG. 11 is a simplified diagram of a section of an embodiment of an implantable lead illustrating how conductors are routed in the implantable lead;

FIG. 12 is a simplified diagram of a cross-section of another embodiment of an implantable lead illustrating how conductors are routed through lumens in the implantable lead;

FIG. 13 is a simplified diagram of an embodiment of a monitoring system including an implantable medical device and an external monitor device;

FIG. 14 is a simplified diagram of an embodiment of an implantable stimulation device in electrical communication with one or more leads implanted in a patient's heart for sensing conditions in the patient, delivering therapy to the patient, or providing some combination thereof; and

FIG. 15 is a simplified functional block diagram of an embodiment of an implantable cardiac device, illustrating basic elements that may be configured to sense conditions in the patient, deliver therapy to the patient, or provide some combination thereof.

In accordance with common practice, the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

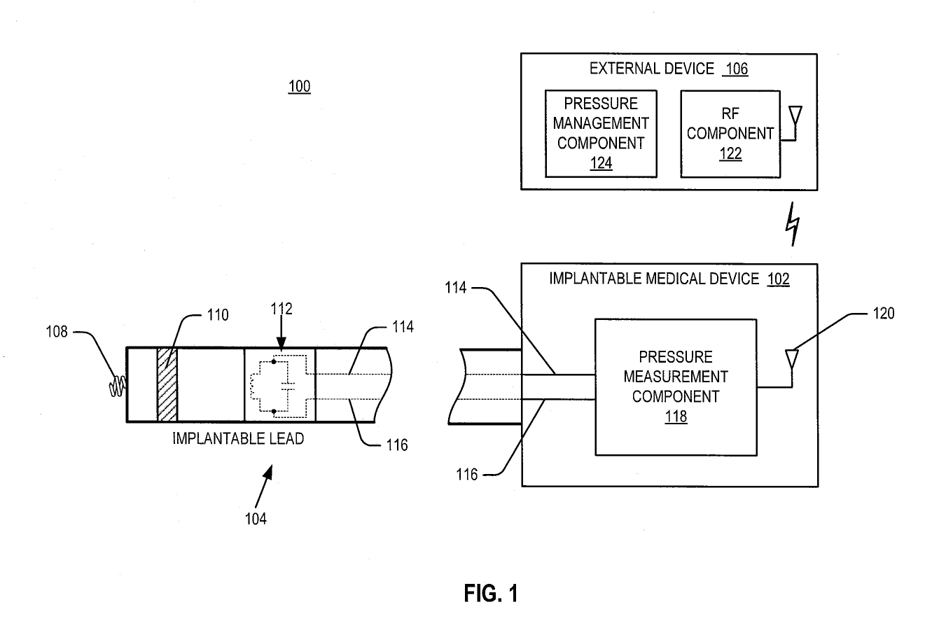

FIG. 1 is a simplified diagram of an embodiment of a medical system 100 where an implantable medical device 102 and an implantable lead 104 are configured to perform pressure measurement operations to provide pressure-related data to an external device 106. To reduce the complexity of FIG. 1, this figure primarily depicts several components (from a high-level perspective) that may be used to acquire pressure information. It should be appreciated, however, that the lead 104 and the devices 102 and 106 will include other components that are used for other medical applications. For example, the lead 104 includes electrodes 108 and 110 and optionally other components that are coupled to circuitry of the device 102 for stimulating cardiac tissue and/or monitoring cardiac activity.

The lead 104 includes an L-C resonant circuit 112 that in operation has a resonant frequency that depends on the pressure in the vicinity of the L-C resonant circuit 112. For example, cardiac pressure exerted an exterior surface of the lead 104 may be coupled via a flexible component (e.g., a flexible insulator material) of the body of the lead 104 to a flexible capacitive circuit of the L-C resonant circuit 112. For purposes of illustration, the L-C resonant circuit 112 is depicted as a parallel inductive-capacitive circuit. This circuit is shown in phantom to indicate that these components reside entirely or at least partially within a lead body of the lead 104. The values of these inductive and capacitive components are selected to cause the L-C resonant circuit 112 to resonate at a specified nominal frequency. As represented by electrical conductors 114 and 116, the L-C resonant circuit 112 is coupled to the device 102 (e.g., via at least one connector as discussed in more detail below) to enable excitation signals to be coupled to the L-C resonant circuit 112 and, upon excitation, to enable the frequency of the L-C resonant circuit 112 to be measured.

The device 102 includes a pressure measurement component 118 that controls excitation of the L-C resonant circuit 112. In particular, the pressure measurement component 118 couples an excitation signal to the L-C resonant circuit 112 via the conductors 114 and 116.

In some embodiments, the pressure measurement component 118 couples an excitation signal received via an antenna to the L-C resonant circuit 112. For example, the pressure measurement component 118 may include a switch that is used to couple the antenna 120 to the conductors 114 and 116 whenever it is desired to conduct a pressure measurement. Consequently, the pressure measurement component 118 may couple a radio frequency (RF) signal generated by the external device 106 (e.g., generated by an RF component 122) or some other external device to the L-C resonant circuit 112. As discussed herein, this RF signal will have a frequency at or near the resonant frequency of the L-C resonant circuit 112 to induce resonant oscillation in the L-C resonant circuit 112.

In other embodiments, the pressure measurement component 118 generates the excitation signal. For example, the pressure measurement component 118 may include a signal generator that generates a signal having a frequency at or near the resonant frequency of the L-C resonant circuit 112.

When the L-C resonant circuit 112 is excited (e.g., induced with a signal that causes the L-C resonant circuit 112 to resonate), an oscillating signal at the resonant frequency is established in the L-C resonant circuit 112. Typically, the oscillating signal resulting from excitation of the L-C resonant circuit 112 will be a damping signal (i.e., decreasing in amplitude over time) since excitation signals are generally not applied to the L-C resonant circuit 112 on a continuous basis.

The pressure measurement component 118 is configured to receive this oscillating signal from the excited L-C resonant circuit 112 via the conductors 114 and 116. In particular, the pressure measurement component 118 processes this received signal to generate data that is representative of the pressure external to the implantable lead 104. The pressure measurement component 118 will then send this data via RF signaling to the external device 106 (e.g., to a pressure management component 124) or some other external device. In this way, an attending physician, clinician, or other personnel may track cardiac pressure (or some other desired pressure) of a patient over time.

The above signal processing may be implemented in various ways. In particular, different aspects of the signal processing may be performed by the device 102 or the device 106 in different embodiments. For example, in some embodiments, the pressure measurement component 118 simply samples the received signal to generate sample data and sends the sample data to the external device 106 for subsequent processing. In some embodiments, the pressure measurement component 118 samples the received signal and processes the samples to determine the frequency of the received signal. In this case, the pressure measurement component 118 may generate data representative of the calculated frequency and send this data to the external device 106 for subsequent processing. In some embodiments, the pressure measurement component 118 calculates a pressure value based on the determined frequency. In this case, the pressure measurement component 118 generates data representative of the calculated pressure sends this data to the external device 106.

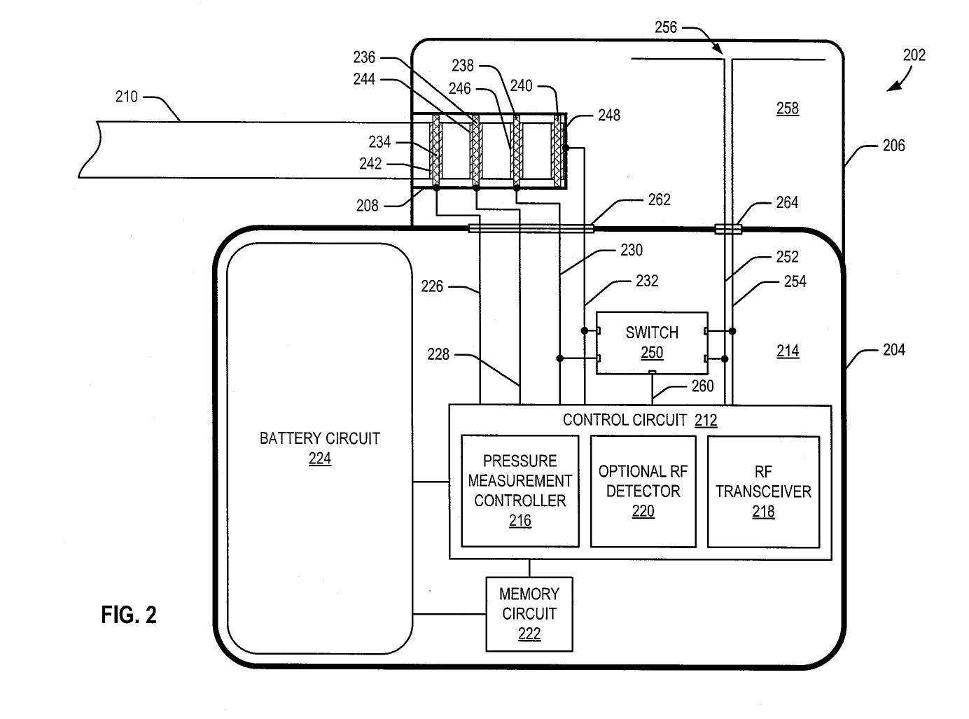

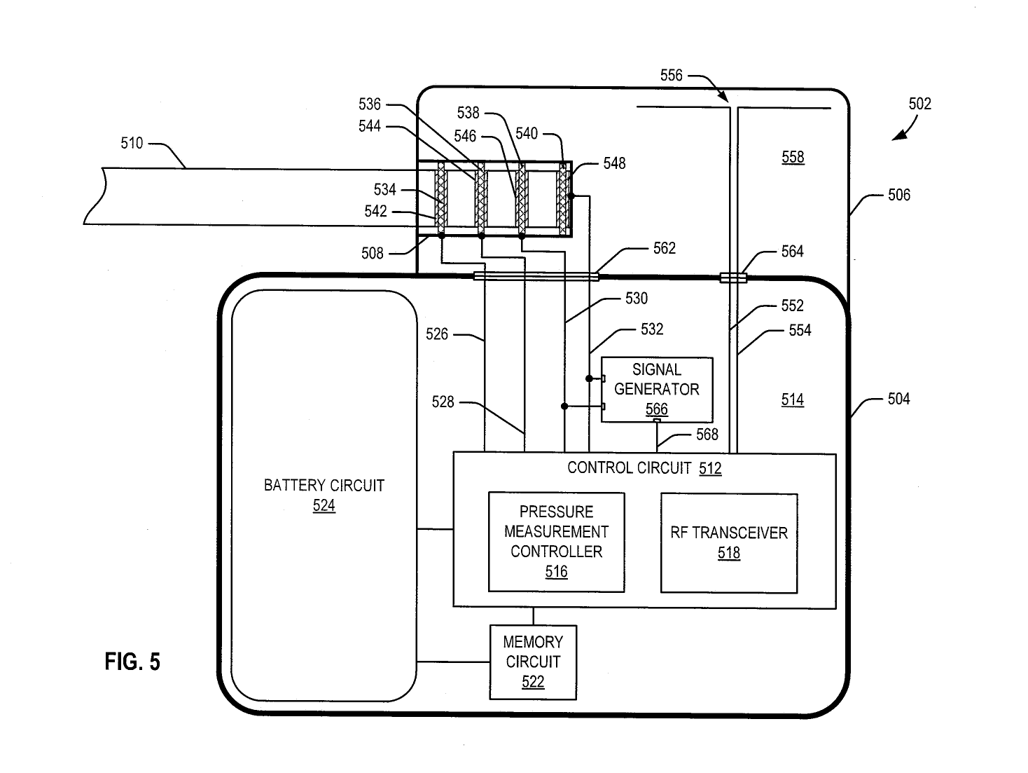

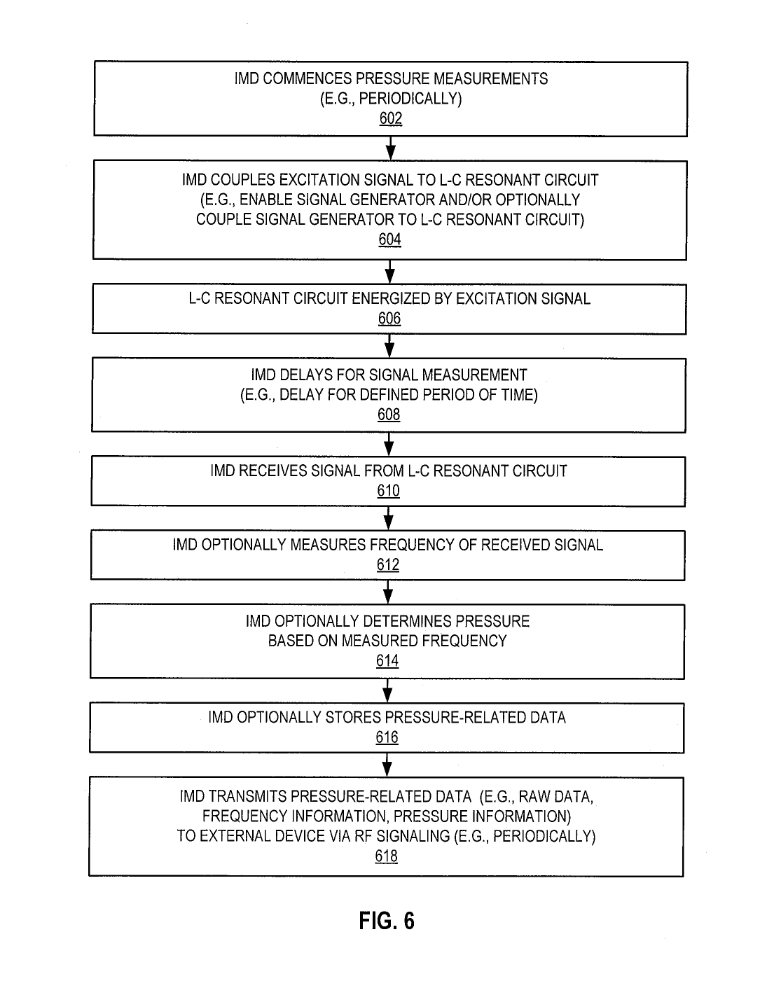

As mentioned above, the device 102 may couple an externally generated excitation signal or an internally generated excitation signal to the L-C resonant circuit 112. FIGS. 2-4 illustrate examples of circuitry and operations that may be employed for the first of these two configurations. FIGS. 5 and 6 illustrate examples of circuitry and operations that may be employed for the second of these two configurations.

In FIG. 2, an implantable medical device 202 includes a housing 204 and a header 206 attached to the housing 204. The header 206 includes a connector 208 that is operable to interface with an implantable lead 210 comprising a passive L-C resonant circuit (not shown in FIG. 2).

A control circuit 212 located within an interior space 214 of the housing 204 performs pressure measurement-related operations as discussed herein. In particular, the control circuit 212 couples an excitation signal to the L-C resonant circuit of the lead 210 and processes signals received from the L-C resonant circuit.

In practice, the control circuit 212 and/or some other circuit (not shown) of the device 202 includes functionality for performing cardiac-related operations (e.g., cardiac sensing and/or pacing operations). To reduce the complexity of FIG. 2, this functionality is not shown.

For purposes of illustration, the control circuit 212 is depicted as including a pressure measurement controller 216, an RF transceiver 218, and an optional RF detector 220 (which could also be implemented as part of the RF transceiver 218); while the device 202 is depicted as including a memory circuit 222, a battery circuit 222, and four conductors 226, 228, 230, and 232. It should be appreciated that different combinations of these components, different quantities of these components, and other components may be employed in other embodiments constructed in accordance with the teachings herein.

The connector 208 includes four terminals 234, 236, 238, and 240, each of which is coupled to a respective one of the conductors 226, 228, 230, and 232. The connector terminals 234, 236, 238, and 240 are operable (e.g., positioned and sized) to couple with corresponding lead terminals located at a proximal section of a lead body. In the example of FIG. 2, lead terminals 242, 244, 246, and 248 of the lead 210 are coupled to corresponding conductors (not shown) of the lead 210 that terminate at corresponding lead circuitry (e.g., the L-C resonant circuit and lead electrodes located at a distal section of the lead body, not shown). Accordingly, the connector 208 facilitates coupling of the L-C resonant circuit and other lead circuitry to the control circuit 212. Again, it should be appreciated that a different number of terminals, a different number of connectors, and different types of connectors may be employed in different embodiments.

For purposes of explanation, the lead terminals 246 and 248 are designated as being coupled to the L-C resonant circuit (e.g., the lead terminals 246 and 248 are coupled to the lead conductors 114 and 116 of FIG. 1). Consequently, the conductors 230 and 232 serve to couple the L-C resonant circuit to the control circuit 212 and a switch 250.

The switch 250 includes several terminals (represented by small rectangles in FIG. 2), each of which is coupled to a corresponding conductor. Specifically, two switch terminals (left side of the switch 250) are coupled to the conductors 230 and 232, another two switch terminals (right side of the switch 250) are coupled to conductors 252 and 254 that are, in turn, coupled to an antenna 256. In this example, the antenna 256 has a dipole configuration and is at least partially located with an interior space 258 defined by the header 206. It should be appreciated, however, that different types of antennas and/or different antenna orientations may be employed in different embodiments.

The switch 250 is operable to selectively couple and decouple pairs of switch terminals based on a control signal received via a control terminal. In the example of FIG. 2, the control circuit 212 supplies this control signal via a conductor 260. For example, when the control signal is a value that causes the switch 250 to be closed, the conductor 230 is coupled to the conductor 252 and the conductor 232 is coupled to the conductor 254. Conversely, when the control signal is a value that causes the switch 250 to be open, the conductor 230 is isolated from the conductor 252 and the conductor 232 is isolated from the conductor 254. Accordingly, under the control of the control circuit 212, the antenna 256 may be selectively coupled to the L-C resonant circuit of the lead 210 to enable a received RF excitation signal (e.g., generated by the external device 106 of FIG. 1) to be coupled to the L-C resonant circuit.

As discussed herein, a change in pressure external to the lead 210 will result in a change in the capacitance of the capacitive circuit of the L-C resonant circuit of the lead 210. This change in capacitance, in turn, causes a change in the resonant frequency of the L-C resonant circuit. Thus, upon excitation of the L-C resonant circuit, the current frequency of an oscillating signal generated by the L-C resonant circuit will correspond to the pressure at the lead 210.

This oscillating signal is detected by the control circuit 222. The control circuit 212 may process the received signal to determine at least one frequency of the signal. Consequently, based on the frequency of the received signal, the control circuit 212 may generate data representative of the pressure external to the lead 210. For example, the generated data may comprise at least one pressure value that was determined based on the determined at least one frequency.

The control circuit 212 may then store this data in the memory circuit 222 for subsequent use. For example, as discussed below, the device 202 may collect data over a period of time, and send the stored data to an external device (not shown in FIG. 2) at some later point in time. As another example, one or more operating parameters (e.g., pacing parameters) of the device 202 may be adapted based on the pressure data.

To facilitate receiving the oscillating signal, the control circuit 212 or interface circuitry (not shown in FIG. 2) of the device 202 may comprise one or more of: a sensing circuit, an amplifier, a filter, a switching circuit, or other suitable circuits. For example, the control circuit 212 may include a high impedance sense amplifier, a low impedance current sensing circuit, or some other suitable receive circuit. Thus, such a circuit may perform one or more of: detecting, filtering, or amplifying the oscillating signal.

As represented by corresponding lines in FIG. 2, the battery circuit 224 is electrically coupled to one or more of the circuits 212 and 222 and any other circuits (not shown) that require power from the battery circuit 224. It should be appreciated that the battery circuit 224 may be implemented using any suitable implantable power source.

As mentioned above, the control circuit 212 is also electrically coupled to the conductors 226 and 228. Thus, the control circuit 212 may be electrically coupled to electrodes (not shown) of the lead 210 for sensing cardiac activity and/or stimulating cardiac tissue. In some cases, these electrodes are used for stimulating cardiac tissue. In some cases, one or more of these electrodes may be used for sensing cardiac activity (e.g., for near-field sensing and/or far-field sensing). To facilitate interfacing with these components, the control circuit 212 and/or other circuitry of the device 202 may comprise one or more of: a sensing circuit, an amplifier, a filter, a signal generator, a signal driver, a switching circuit, or other suitable circuits.

The control circuit 212 may process cardiac signals received via the lead 210 to identify cardiac events. For example, a microprocessor of the control circuit 212 may be configured to acquire intra-cardiac electrogram data (and/or other cardiac related signal data) and identify P waves, R waves, T waves and other cardiac events of interest. Based on analysis of these cardiac events, the processing circuit may selectively generate stimulation signals (e.g., pacing pulses) to be delivered to cardiac tissue via one or more electrodes.

The control circuit 212 also may control stimulation operations. For example, a microprocessor of the control circuit 212 may be configured to trigger the generation of pacing signals, specify pacing signal characteristics (e.g., energy level and duration), and inhibit pacing signals.

It should be appreciated that the control circuit 212 may take various forms in different embodiments. For example, in some implementations, a single circuit (e.g., a microprocessor) may be employed to handle processing for both pressure sensing and cardiac operations. In other implementations, however, different circuits may be employed to provide the processing for these different operations.

In practice, the device 202 is biocompatible and hermetically sealed. For example, the housing 204 may be constructed of a material such as titanium. In addition, the header 206 may be constructed of a material such as silicone. The device 202 also includes hermetically sealed feedthroughs for routing conductors between the interior space 214 of the housing and the interior space 258 of the header 206. In the example of FIG. 2, the conductors 226, 228, 230, and 232 are routed through a feedthrough 262 and the conductors 252 and 254 are routed through a feedthrough 262. Typically, signal filtering is provided at each feedthrough to prevent undesirable signals (e.g., RF interference, magnetic resonance imaging (MRI) signals, etc.) from entering the housing 204 and adversely affecting the operation of the circuitry of the device 202. For the conductors 230, 232, 252, and 254 that carry the excitation signal, the filtering for these feedthroughs is designed to allow passage of signals near the nominal resonant frequency of the L-C resonant circuit.

It should be appreciated that a given implementation may incorporate some of all of the functionality discussed herein. For example, in some embodiments, an external device may directly receive the oscillating signal generated by the LC-resonant circuit. In such a case, the device 202 need not employ circuitry for receiving a signal from the L-C resonant circuit or for generating data representative of cardiac pressure. Rather, based on the measurements made by the external device, the external device will determine the cardiac pressure in these embodiments. Conversely, in some embodiments, an external device may directly excite the LC-resonant circuit. In such a case, the device 202 need not employ circuitry for coupling an excitation signal to the L-C resonant circuit.

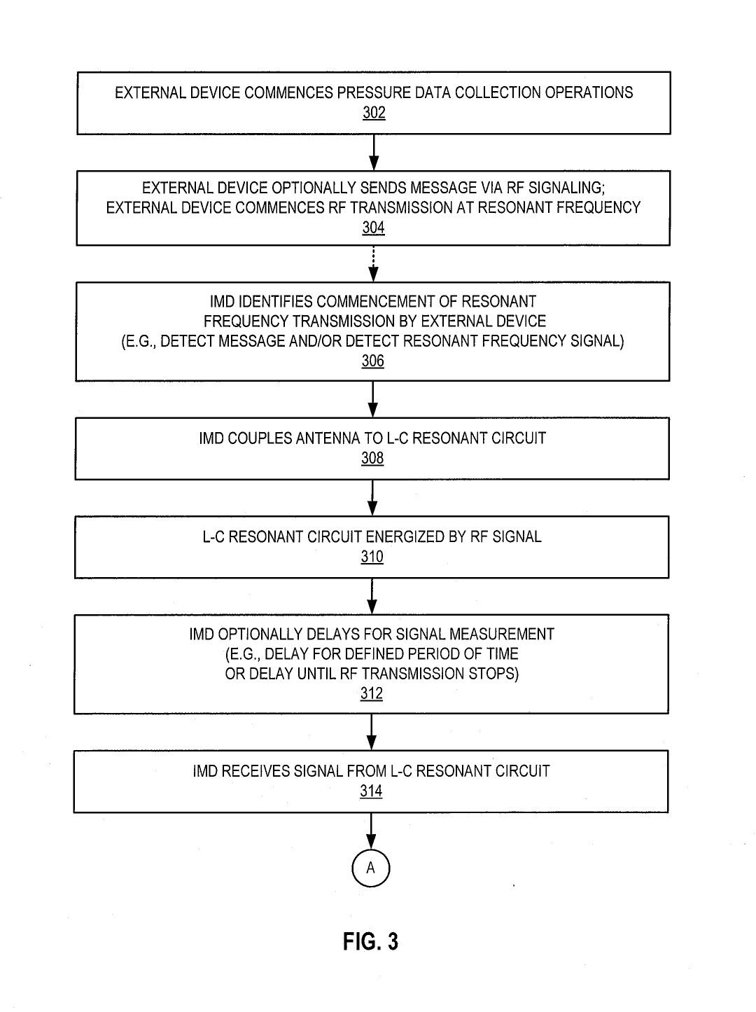

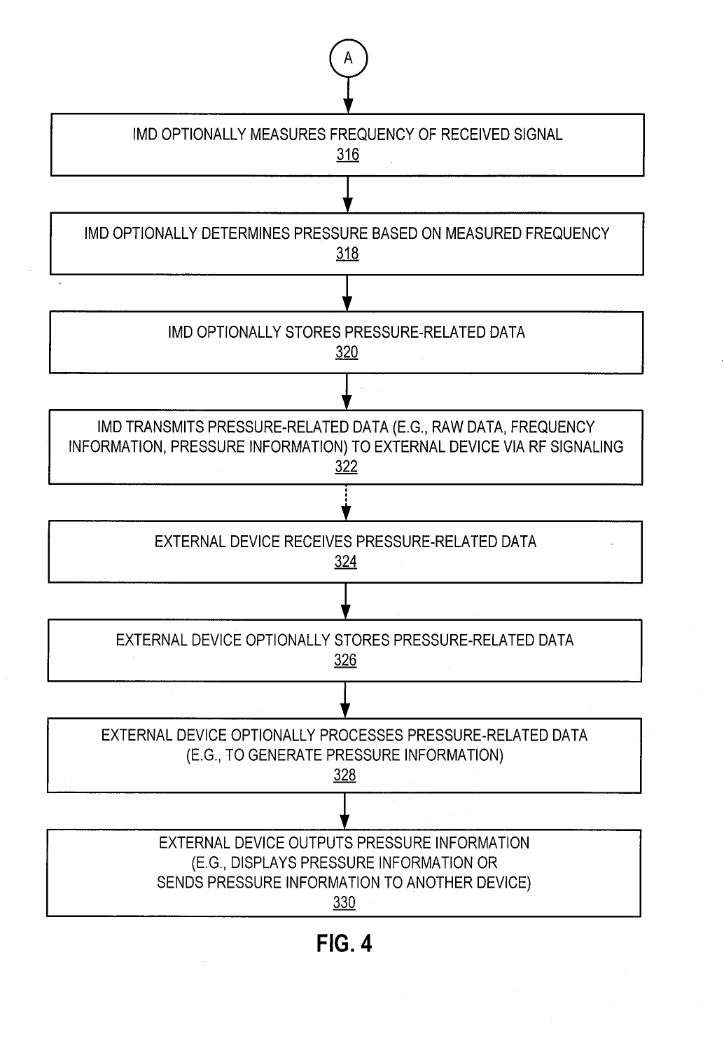

With the above in mind, a more detailed example of operations that may be performed to conduct pressure measurements based on an external excitation signal will be described with reference to the flowchart of FIGS. 3 and 4. For convenience, the operations of FIGS. 3 and 4 (or any other operations discussed or taught herein) may be described as being performed by specific components. It should be appreciated, however, that these operations may be performed by other types of components and may be performed using a different number of components. It also should be appreciated that one or more of the operations described herein may not be employed in a given implementation.

As represented by block 302 of FIG. 3, at some point in time, an external device commences pressure data collection operations. For example, a user (e.g., a doctor, a clinician, a patient, etc.) of the external device may initiate this operation or the external device may be configured to perform these operations at designated times (e.g., periodically, at nighttime, etc.).

As represented by block 304, in conjunction with commencing the pressure data collection operation, the external device sends one or more signals to an implantable medical device (IMD) implanted in a patient to inform the IMD that it should commence pressure measurement operations.

In some implementations, this RF signaling involves the external device sending a message to IMD. This message may take different forms in different embodiments. In some embodiments, the message indicates that the external device will commence transmission of an RF signal having a frequency substantially equal to a nominal resonant frequency of the L-C resonant circuit. In some embodiments, the message comprises a request to close or open the switch at the IMD. In either case, after transmitting the message, the external device (or another external device) commences transmitting an RF signal having a frequency substantially equal to a nominal resonant frequency of the L-C resonant circuit.

In some implementations, instead of sending a message, the external device simply commences transmitting an RF signal having a frequency substantially equal to a nominal resonant frequency of the L-C resonant circuit to begin the pressure measurement operation. This approach may be used, for example, in a case where the IMD is configured to regularly monitor for RF signals in this frequency range.

Blocks 306-322 describe several operations that may be performed by an IMD that received the signals transmitted by the external device.

As represented by block 306, the IMD identifies commencement of resonant frequency transmission by the external device. This operation may involve determining that such transmission has already commenced or determining that such transmission will occur (e.g., is scheduled to commence).

In some implementations, the operations of block 306 involve detecting a message transmitted by an external device. Based on this detection, the IMD may generate a control signal to control whether a switch is opened or closed.

To this end, the IMD may include an RF transceiver operable to receive messages via an antenna. In addition, the IMD may include a control circuit (e.g., a microprocessor) operable to decode received messages. For example, the IMD may decode a received message to determine whether the message indicates that an external device will commence transmission of an RF signal having a frequency substantially equal to a nominal resonant frequency of the L-C resonant circuit. As another example, the IMD may decode a received message to determine whether an external device has sent a request to close or open a switch at the IMD.

In some implementations, the operations of block 306 involve detecting a resonant frequency signal. For example, the IMD may include an RF detector circuit operable to detect an RF signal having a frequency substantially equal to a nominal resonant frequency of the L-C resonant circuit. In addition, the IMD may include a control circuit (e.g., a microprocessor) operable to generate a control signal based on whether the RF detector circuit detects such an RF signal. As discussed herein, this control signal may be generated to control whether a switch is opened or closed.

As represented by block 308, based on the identification of an RF transmission at block 306, the IMD couples its antenna to the L-C resonant circuit of an implantable lead connected to the IMD. For example, the IMD may generate a control signal for controlling a switch coupled between the antenna and a lead connector of the IMD, and this control signal may be generated based on whether an RF signal is received at the antenna (e.g., is currently being received or is expected to be received).

As represented by block 310, the L-C resonant circuit of the lead is energized by an RF signal received by the IMD. For example, in FIG. 2, an RF signal received via the antenna 256 is coupled by the switch 250 from the conductors 252 and 254 to the conductors 230 and 232. The RF signal is thus coupled from the connector terminals 238 and 240 to the lead terminal 246 and 248 and then to the L-C resonant circuit via corresponding lead conductors (not shown).

As represented by block 312, the IMD may optionally invoke a delay prior to commencing signal measurement operations. In this way, the IMD may insure that the L-C resonant circuit has been excited and has achieved resonant oscillation. For example, the control circuit may generate the control signal in a manner that ensures that the switch is closed for a defined period of time. As another example, the control circuit may generate the control signal in a manner that ensures that the switch is closed for as long as the RF signal is being received.

As represented by block 314, the IMD monitors for an oscillating signal from the excited L-C resonant circuit. Referring again to the example of FIG. 2, after the switch 250 is opened (or the RF signal is longer being received), the control circuit 212 or an interface circuit (e.g., a receiver) may receive an oscillating signal from the L-C resonant circuit via the conductors 230 and 232.

The IMD may then process the received signal to generate data representative of pressure induced on the lead. For example, a control circuit or an interface circuit may sample a received oscillating signal to generate digital data. Blocks 316 and 318 of FIG. 4 illustrate two other examples of signal processing operations.

As represented by block 316, in some embodiments, the IMD may measure at least one frequency (e.g., a dominant frequency, a center frequency, etc.) of the received signal. For example, the IMD may process sampled data to generate data representative of a frequency of the signal.

As represented by block 318, in some embodiments, the IMD may determine the pressure external to the lead based on the at least one frequency determined at block 316. For example, the IMD may process the data representative of a frequency of the signal to determine the pressure that corresponds to the L-C resonant circuit generating an oscillating signal at this frequency.

As represented by block 320, the IMD may store any of the pressure-related data generated at one or more of blocks 314, 316, or 318. For example, the IMD may collect the data over a period of time for subsequent (e.g., periodic) reporting.

As represented by block 322, the IMD transmits the pressure-related data to an external device via RF signaling. Referring to the example of FIG. 2, the RF transceiver 218 may be operable to communicate via the antenna 256 with an external device to send the data generated by the IMD (e.g., raw sample data, frequency information, or pressure information).

Blocks 324-330 describe several operations that may be performed by an external device that receives the information transmitted by the IMD at block 322. As represented by block 324, this information is received at the IMD, for example, by probing the IMD, via scheduled uploads, or using some other communication operation. The external device optionally stores the received information as represented by block 326.

As represented by block 328, the external device may process the received pressure-related information. For example, in cases where the pressure-related information comprises raw sample data or an indication of the frequency of the oscillating signal, the external device may process this data to generate pressure information indicative of the pressure external to the implanted lead.

As represented by block 330, the external device outputs pressure information based on the received pressure-related information. For example, the external device may display an indication of the pressure on a display device. As another example, the external device may send the pressure information to another device (e.g., a networked device) to enable users to access the pressure information via that device.

The pressure information may be used in various ways. For example, applications include heart failure (HF) monitoring and interventions (e.g., AV, VV delays, paired pacing, and so) by measuring RV dP/dt and estimated PAP. Paired pacing involves producing two electrical beats but only one mechanical beat. It is also referred as coupling interval of paired pacing pulses for electrical-mechanical dissociation of the second pacing pulse. The approach could affect contractility that could be measured through the use of apparatuses and methods implemented according to the teachings herein.

Referring now to FIG. 5, in this embodiment, an implantable medical device 502 includes a signal generator 566 operable to output an excitation signal for an L-C resonant circuit (not shown) of an implantable lead 510 connected to the device 502. This excitation signal has a frequency substantially (e.g., exactly or approximately) equal to a nominal resonant frequency of the L-C resonant circuit.

Thus, in this embodiment, the L-C resonant circuit is excited by an internal (relative to the IMD) excitation circuit instead of by external excitation signals. Specifically, the signal generator 566 generates a signal (e.g., a single pulse, a set of pulses, or a periodic pulse signal) that is provided to the L-C resonant circuit to excite the L-C resonant circuit and, if applicable, maintain oscillations in the L-C resonant circuit.

The control circuit 512 (or some other suitable circuit of the device 302) controls the operation of the signal generator 566. For example, upon receipt of a suitable message from an external device (e.g., a pressure measurement command from an external monitoring device) at the control circuit 512, the control circuit 512 may generate a control signal that causes the signal generator 566 to commence excitation of the L-C resonant circuit. As another example, the control circuit 512 may be configured to initiate excitation at certain times (e.g., periodically).

The signal generator 566 includes several terminals (represented by small rectangles in FIG. 5), each of which is coupled to a corresponding conductor. Specifically, two output terminals (left side of the signal generator 566) are coupled to conductors 530 and 532. In this example, the conductors 530 and 532 are designated as being coupled to the L-C resonant circuit (e.g., lead terminals 546 and 548 are coupled via lead conductors (not shown) to the L-C resonant circuit).

The signal generator 566 is operable to selectively output an excitation signal on the conductors 530 and 532 based on a control signal received via a control terminal (bottom side of the signal generator 566). In the example of FIG. 5, the control circuit 512 (e.g., the pressure measurement controller 516) supplies this control signal via a conductor 568.

Thus, the control circuit 512 generates the control signal to selectively control whether the excitation signal is provided to the L-C resonant circuit. For example, the control circuit 512 may generate the control signal based on a determination of whether a received message (decoded by the control circuit) indicates that an external device has sent a request to conduct a pressure measurement.

Subsequent to the above excitation operations, the control circuit 512 receives an oscillating signal from the L-C resonant circuit. The control circuit 512 then processes this received signal to generate data representative of pressure induced on the implantable lead 510. For example, the control circuit 512 may process the received signal to determine at least one frequency of the signal. In addition, the control circuit 512 may generate at least one pressure value based on the determined at least one frequency. Consequently, the control circuit 512 may generate data representative of the pressure external to the lead 510 in a similar manner as discussed above.

The control circuit 512 may then store this data in the memory circuit 522 for subsequent use. For example, as discussed below, the device 502 may collect data over a period of time, and send the stored data to an external device (not shown in FIG. 5) at some later point in time. As another example, one or more operating parameters (e.g., pacing parameters) of the device 502 may be adapted based on the pressure data.

The device 502 comprises a housing 504, a header 506, and other circuitry similar to the device 202 of FIG. 2. In particular, components of FIG. 5 that have similar reference numbers as components of FIG. 2 (i.e., 5xx versus 2xx) may have similar functionality. For purposes of brevity, a discussion of these similar components will not be repeated.

With the above in mind, a more detailed example of operations that may be performed to conduct pressure measurements based on an internal excitation signal will be described with reference to the flowchart of FIG. 6.

As represented by block 602, at some point in time, an implantable medical device (IMD) implanted in a patient commences pressure measurements. For example, in some cases, the operations of block 602 involve detecting a message transmitted by an external device. To this end, the IMD may include an RF transceiver operable to receive messages via an antenna. In addition, the IMD may include a control circuit (e.g., a microprocessor) operable to decode received messages. For example, the IMD may decode a received message to determine whether the message indicates that an external device has sent a request to conduct a pressure measurement. As another example, in some cases, the operations of block 602 involve periodically invoking pressure measurement operations.

As represented by block 604, upon commencement of the pressure measurement, the IMD couples an excitation signal to an L-C resonant circuit of an implantable lead connected to the IMD. For example, the control circuit of the IMD may generate a control signal to control whether a signal generator is enabled and/or to couple the output of the signal generator to the L-C resonant circuit. As discussed above, this control signal may be generated based on a determination of whether a message received from an external device indicates that the external device has sent a request to conduct a pressure measurement.

As represented by block 606, the L-C resonant circuit of the lead is thus energized by the excitation signal generated by the IMD. For example, in FIG. 5, a signal generated by the signal generator 566 is coupled to the conductors 530 and 532. Consequently, the signal is coupled from the connector terminals 238 and 240 to the lead terminals 246 and 248 and then to the L-C resonant circuit via corresponding lead conductors (not shown).

As represented by block 608, the IMD may optionally invoke a delay prior to commencing signal measurement operations (e.g., to insure that the L-C resonant circuit has been excited and has achieved resonant oscillation). For example, the control circuit may generate the control signal in a manner that ensures that the switch is closed for a defined period of time.

As represented by block 610, the IMD receives an oscillating signal from the excited L-C resonant circuit. Referring again to the example of FIG. 5, the control circuit 512 or an interface circuit (e.g., a receiver) may receive an oscillating signal from the L-C resonant circuit via the conductors 530 and 532.

The IMD may then process the received signal to generate data representative of pressure induced on the lead. For example, a control circuit or an interface circuit may sample a received oscillating signal to generate digital data. As represented by block 612, in some embodiments, the IMD may measure at least one frequency (e.g., a dominant frequency, a center frequency, etc.) of the received signal. As represented by block 614, in some embodiments, the IMD may determine the pressure external to the lead based on the at least one frequency determined at block 612. As represented by block 616, the IMD may store the generated pressure-related data.

As represented by block 618, the IMD transmits the pressure-related data generated at one or more of blocks 610, 612, or 614 to an external device via RF signaling. Referring to the example of FIG. 5, the RF transceiver 518 may be operable to communicate via the antenna 556 with an external device to send the data generated by the IMD (e.g., raw sample data, frequency information, or pressure information).

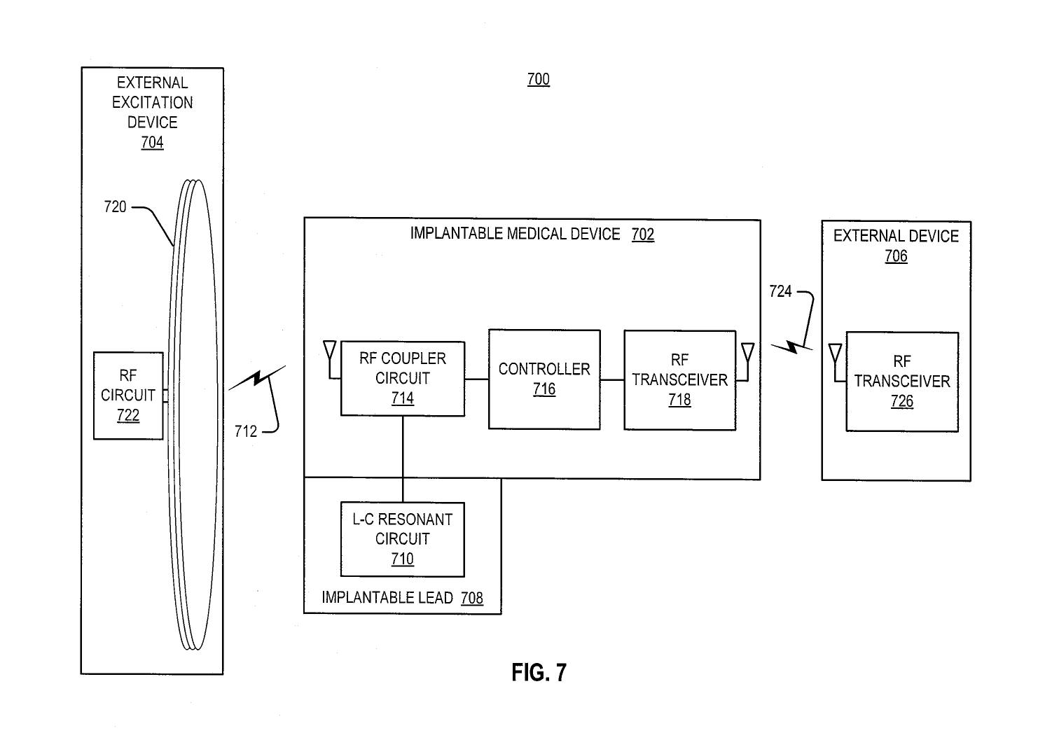

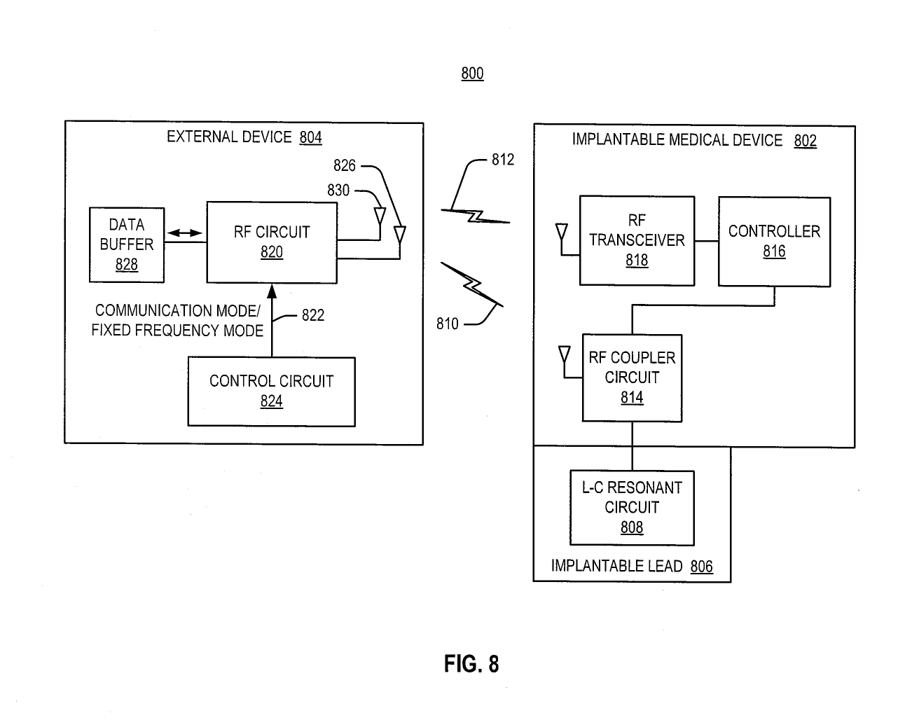

An implantable medical device may communicate with external devices in different ways in different embodiments. FIGS. 7 and 8 depict two examples illustrating how an implantable medical device may communicate with different types of external devices.

FIG. 7 illustrates an embodiment of a system 700 where an implantable medical device 702 that is implanted in a patient (not shown) communicates with an external device 704 and an external device 706. In this example, an implantable lead 708 connected to the device 702 includes an L-C resonant circuit 710 that is excited by RF signals 712 generated by the external device 704. In addition, the device 702 communicates with the external device 706 (e.g., a programmer, a home monitor, etc.) to, for example, upload and download information.

The device 702 includes an RF coupler circuit 714 (e.g., a switch), a controller 716 (e.g., a control circuit), and an RF transceiver 718 that are electrically coupled with one another, as applicable. Several other circuits that would be included in the device 702 (e.g., a battery circuit) are not shown to reduce the complexity of FIG. 7.

The external device 704 includes an antenna 720 (e.g., a coil) that may be much larger than an antenna of the device 702 (e.g., an antenna for the RF coupler circuit 714). For example, the antenna 720 may have dimensions of 12-20 centimeters in diameter while the antenna for the RF coupler circuit 714 may be a few centimeters wide. In this way, an RF circuit 722 of the external device 704 is able to more effectively couple relatively high frequency RF signals 712 through the tissue of a patient (not shown) to the device 702. As discussed herein, the frequency of the RF signals 712 may be at or near a nominal resonant frequency of the L-C resonant circuit 710.

The RF transceiver 718 and associated antenna communicate with the external device 706 via RF signals 724. For example, the external device 706 may communicate with the device 702 to initiate pressure sensing operations, to upload data generated by the pressure sensing operations, to control cardiac-related operations, and so on. Of note, the external device 706 may employ a smaller antenna (not shown) than the antenna 720 since less RF energy may be required to communicate with the device 702 than is required to excite the L-C resonant circuit 710 due to the use of lower frequency RF signals for this communication.

Although FIG. 7 depicts the device 702 as including two different antennas for receiving the RF excitation signal from the external device 704 and communicating with the external device 706, in some implementations a single antenna may be used to handle both types of RF signaling.

FIG. 8 illustrates an embodiment of a system 800 where an implantable medical device 802 that is implanted in a patient (not shown) communicates with an external device 804. In this example, an implantable lead 806 connected to the device 802 includes an L-C resonant circuit 808 that is excited by RF signals 810 generated by the external device 804. In addition, the device 802 communicates with the external device 804 (e.g., a programmer, a home monitor, etc.) via RF signaling 812 to, for example, upload and download information.

Similar to the device 702 of FIG. 7, the device 802 includes an RF coupler circuit 814 (e.g., a switch), a controller 816 (e.g., a control circuit), and an RF transceiver 818 that are electrically coupled in a suitable manner. Several other circuits that would be included in the device 802 are not shown to reduce the complexity of FIG. 8.

The configuration of FIG. 8 may be employed in cases where the external device 804 also includes the capability to excite an L-C resonant circuit. For example, the external device 804 may include an RF circuit 820 that is capable of selectively operating in a fixed frequency mode or a communication mode based on a control signal 822 generated by a control circuit 824.

In the fixed frequency mode of operation, the RF circuit 820 generates an RF signal for exciting the L-C resonant circuit 808. Here, the RF circuit 820 is configured to transmit the RF signal 810 via an antenna 826 (e.g., a loop antenna) so that the RF signals 810 are effectively coupled to an antenna of the RF coupler circuit 814.

In the communication mode of operation, the RF circuit 820 (e.g., comprising an RF transceiver) transmits data from a data buffer 828 and stores received data in the data buffer 828. In this case, the RF circuit 820 is configured to transmit and receive corresponding RF signals 812 via an antenna 830 when communicating with the RF transceiver 818.

In some aspects, the use of the single external device 804 for both operations is enabled based on the teachings herein because relative large reactive components may be employed for the L-C resonant circuit 808. For example, by using a sufficiently large right ventricle lead (e.g., in contrast with a relatively small dedicated passive pressure sensor implanted in the pulmonary artery), larger reactive components may be employed in the L-C resonant circuit 808. As a result, the L-C resonant circuit 808 may be implemented at a lower resonant frequency. Consequently, since a lower frequency RF signal is required in this case, the external device 804 may employ a smaller antenna (e.g., the antenna 826); yet still couple sufficient energy to the device 802 to excite the L-C resonant circuit 808.

Also, although FIG. 8 depicts the device 802 as including two different antennas for receiving the RF excitation signal and communicating with the external device 804, in some implementations a single antenna may be used to handle both types of RF signaling.

In view of the above, an implantable medical device constructed in accordance with the teachings herein may provide one or more advantages over conventional medical systems. For example, such a device may provide sensing and/or pacing along with pressure sensing in a single implantable system. The use of an implantable medical device as taught herein may facilitate using larger pressure sensor components (e.g., capacitor and inductor), thereby enabling the use of a lower resonant frequency which may, in turn, enable the use of a smaller antenna coil at an external device. The use of an implantable medical device enables power (from a battery circuit) to be readily provided for the pressure sensor, provides more effective telemetry for upload and downloading information (e.g., via on-board RF components), and facilitates acquisition of data over a period of time (e.g., via an on-board memory circuit). Moreover, in some embodiments, a single antenna (e.g., a conventional telemetry antenna) may be used to for receiving excitation signals from an external device and communicating with an external device.

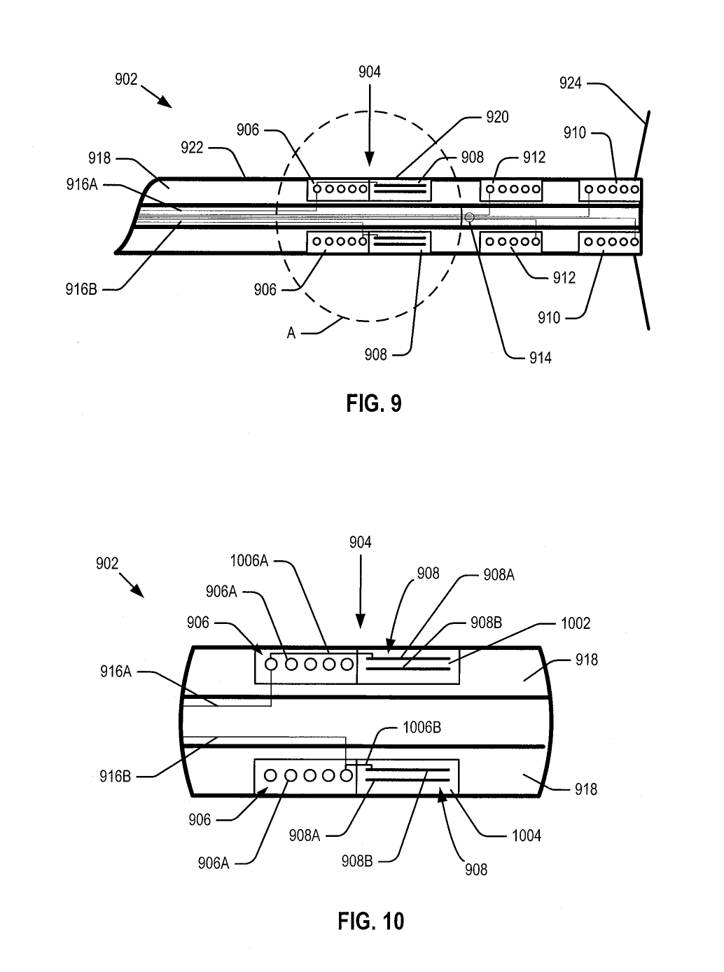

FIG. 9 illustrates, in a simplified sectional side view, an embodiment of an implantable lead 902 that incorporates a passive pressure sensor circuit 904 in accordance with the teaching herein. The pressure sensor circuit 904 comprises an inductive circuit 906 (e.g., a wound inductor) and a capacitive circuit 908 (e.g., a pair of conductive plates separated by a dielectric material).

In this example, the pressure sensor circuit 904 is incorporated into a distal section of the lead 902. It should be appreciated, however, that a pressure sensor circuit 904 may be incorporated into different locations along the length of the lead 902 to facilitate obtaining pressure measurements from different locations within a patient.

FIG. 9 illustrates that in some embodiments the pressure sensor circuit 904 is located adjacent an exterior surface of a biocompatible lead body 918 of the lead 902. In this example, an exterior surface 920 of the pressure sensor circuit 904 is coplanar with the exterior surface 922 of the lead body 918. Thus, the external surface 920 of the pressure sensor circuit 904 would be flexible (e.g., to couple pressure waves to the capacitive circuit 908) and biocompatible in this case. For example, the external surface 920 may comprise silicone or some other flexible biocompatible material. In other embodiments, however, the pressure sensor circuit 904 may be located completely within the lead body 918. In these cases, the pressure sensor circuit need not be biocompatible.

In some implementations, the pressure sensor circuit 904 may be electrically isolated from (i.e., not electrically coupled to) any other electrical components of the lead 902. For example, the lead 902 includes a tip electrode coil 910 and a ring electrode coil 912 that are coupled to four conductors 914. However, the inductive circuit 906, the capacitive circuit 908, and associated electrical conductors 916A and 916B are insulated from the conductors 914 and the coils 910 and 912 (e.g., via insulation material on the conductive materials and/or a gap in the interior of the lead 902).

In the example of FIG. 9, the lead 902 is shown as including a passive fixation element 924. It should be appreciated, however, that a passive pressure sensor circuit as taught herein may be incorporated into an implantable lead employing active fixation or into some other type of implantable lead.

FIG. 10 is an enlarged representation of the view A of FIG. 9. This figure illustrates the connectivity and structure of the pressure sensor circuit 904 in more detail. In particular, FIG. 10 serves to illustrate that the pressure sensor circuit 904 may take the form of an inductive-capacitive (LC) resonant circuit having a cylindrical structure.

As represented by the plates 908A and 908B of the capacitive circuit 908 in FIG. 10, each plate of the capacitive circuit 908 may take the form of a cylinder or a partial cylinder. Here, each cylinder is oriented in a longitudinal direction along the longitudinal axis of the lead body 918. That is the longitudinal axis of each cylinder is parallel with (or, in some cases, the same as) longitudinal axis of the lead body 918. Due to the large plate surface area that this configuration provides, the plates 908A and 908B of the capacitive circuit 908 may be more susceptible to relative deformation when the lead 902 is subjected to changes in external pressure. Consequently, the resonant circuit comprised of the capacitive circuit 908 and the inductive circuit 906 will be more sensitive to pressure changes, thereby facilitating more accurate pressure readings in some cases.

FIG. 10 also illustrates that a relatively flexible dielectric material 1002 (e.g., a fiberglass material) may be disposed between the plates 908A and 908B of the capacitive circuit 908. In this way, external pressure induced on the lead 902 may more easily cause the distance between the plates 908A and 908B to change. Thus, the resonant circuit comprised of the capacitive circuit 908 and the inductive circuit 906 will be more sensitive to pressure changes, thereby facilitating more accurate pressure readings in some cases.

A relatively flexible material 1004 (e.g., a silicone-based material) may be disposed adjacent (e.g., next to or under) an exterior surface of the lead body 918 and engaged with (e.g., disposed against, in contact with, etc.) the capacitive circuit 908. The flexible material 1004 (e.g., a flexible insulator material) may thus serve to couple pressure waves to the capacitive circuit 908 in an efficient manner. As discussed above, in some embodiments (e.g., as shown in FIG. 10), the flexible material 1004 may comprise a portion of the outer surface of the lead. In this case, the flexible material 1004 itself will form part of the hermetic seal for the lead 902, along with hermetic sealing (e.g., via adhesive or welding) between the flexible material 1004 and lead body 918. For example, a thin layer of fiberglass (or some other suitable material) may be provided over an outer enclosure of the capacitive circuit 908 (or directly over an outer plate of the capacitive circuit 908).

In other embodiments (not shown in FIG. 10), the flexible material 1004 may be housed entirely within (but located adjacent to) the lead body 918. In such a case, the biocompatible lead body 918 may provide the hermitic seal. In addition, the lead body 918 will be sufficiently flexible here to couple pressure waves to the capacitive circuit 908 (e.g., via the flexible material 1004). For example, the lead body 918 may comprise a relatively thin outer layer (e.g., constructed of silicone, fiberglass, or some other suitable material) that covers an outer enclosure of the capacitive circuit 908 (or covers an outer plate of the capacitive circuit 908).

As represented by the conductor 906A of the inductive circuit 906 in FIG. 10, the inductive circuit 906 may take the form of a cylindrical coil or some other coil-like structure. For example, the coil conductor may start at the upper left circle of FIG. 10 (connected to a conductor 1006A) and wrap around the interior of the lead 902, terminating at the lower right circle of FIG. 10 (connected to a conductor 1006B).

The inductive circuit 906 may be constructed in various ways. In some embodiments, the inductive circuit 906 is constructed on a PEEK bobbin with DFT wire (41% AG or less) or copper wire. The wire may be coated with, for example, ETFE or some other insulation material. In some embodiments, the wire may be relatively thin (e.g., 100 micrometers to 2 mils) so that the coil may have large number of turns, thereby providing a higher value of inductance for a given size coil.

FIGS. 9 and 10 illustrate an embodiment where the inductive circuit 906 and the capacitive circuit 908 are physically located in a series relationship with respect to one another (i.e., one circuit is positioned further down the lead body 918 from the other circuit). In other embodiments (not shown), the capacitive circuit 908 may be located over the inductive circuit 906. That is, the inductive circuit 906 and the capacitive circuit 908 may have a concentric relationship with one another.

As FIG. 10 illustrates, one terminal of the inductive circuit 906 is coupled via the conductor 1006A to the plate 908A of the capacitive circuit 1008, while the other terminal of the inductive circuit 906 is coupled via the conductor 1006B to the plate 908B of the capacitive circuit 908. Thus, the inductive circuit 906 and the capacitive circuit 908 are coupled in parallel, thereby forming a passive resonant circuit that is capable of being excited by an externally applied electromagnetic field.

The physical properties of the inductive circuit 906 (e.g., the number of turns) and the capacitive circuit 908 (e.g., size and distance between plates) are selected to provide a desired resonant frequency for the sensor circuit 904. In some embodiments, the resonant circuit has a resonant frequency of 35 MHz or less (e.g., 30 MHz). Such a circuit may be compatible with other types of passive pressure sensors.

In some embodiments, the resonant circuit has a resonant frequency of 20 MHz or less (e.g., 10-15 MHz). This lower resonant frequency may be achieved, for example, as a result of the physical characteristics (e.g., the size and shape) of the passive pressure sensor that can be achieved in an implantable device based on the teachings herein. Such a circuit may advantageously enable the use of a smaller transmission coil at the external monitoring system or other similar device. Consequently, a more portable external monitoring system (or other device) may be employed to acquire pressure readings from a passive pressure sensor constructed in accordance with the teachings herein. Alternative, this smaller size may enable the transmission coil to be incorporated into an external device (e.g., a programmer) used for communicating with an implantable medical device (e.g., a pacemaker, an ICD, etc.).

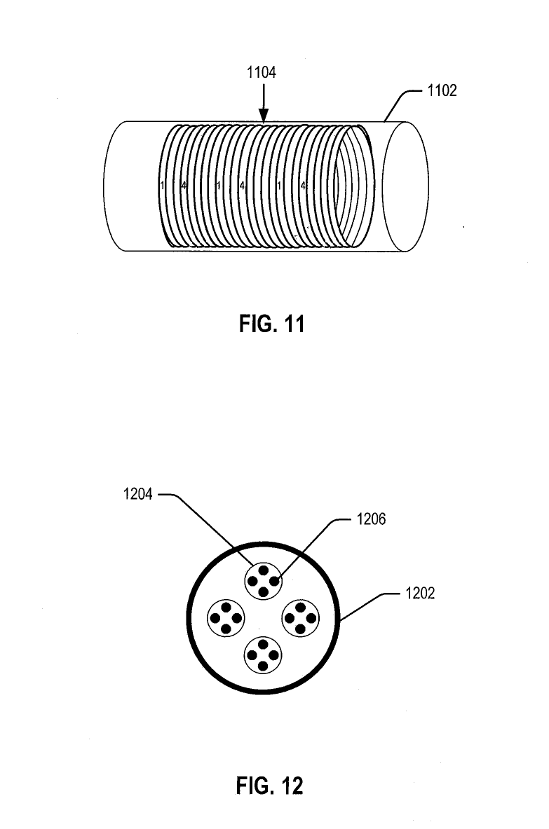

FIGS. 11 and 12 illustrate two examples of how electrical conductors coupled to an L-C resonant circuit may be routed through an implantable lead.

In FIG. 11, a lead 1102 includes redundant lead conductors 1104 routed in a coaxial manner near the exterior circumference of the lead 1102. Such a lead may comprise, for example, a bradycardia lead. In the simplified example of FIG. 11, eight different lead conductors 1-8 are illustrated, with lead conductors 1 and 4 labeled. To accommodate an L-C resonant circuit in the lead 1102, one or more of the redundant lead conductors (e.g., lead conductors 1 and 4) may be re-designated for coupling the L-C resonant circuit to a lead connector (not shown).

FIG. 12 illustrates a cross-section view of a lead 1202 that includes lumens 1204 for routing lead conductors 1206 along a longitudinal axis of the lead 1202. Such a lead may comprise, for example, a tachycardia lead. In the simplified example of FIG. 11, four different lumens 1204 are illustrated. To accommodate an L-C resonant circuit in the lead 1202, one or more of the lumens 1204 may be used for routing a lead conductor that couples the L-C resonant circuit to a lead connector (not shown).

It should thus be appreciated that electrical conductors coupled to an L-C resonant circuit may be routed through an implantable lead in various ways. As another example, a lead may include lead conductors routed in a co-radial manner. Such a co-radial lead may be implemented, for example, as a passive fixation lead with all coils at the same radius. In some cases, co-radial leads are advantageously employed in MRI-compatible applications.

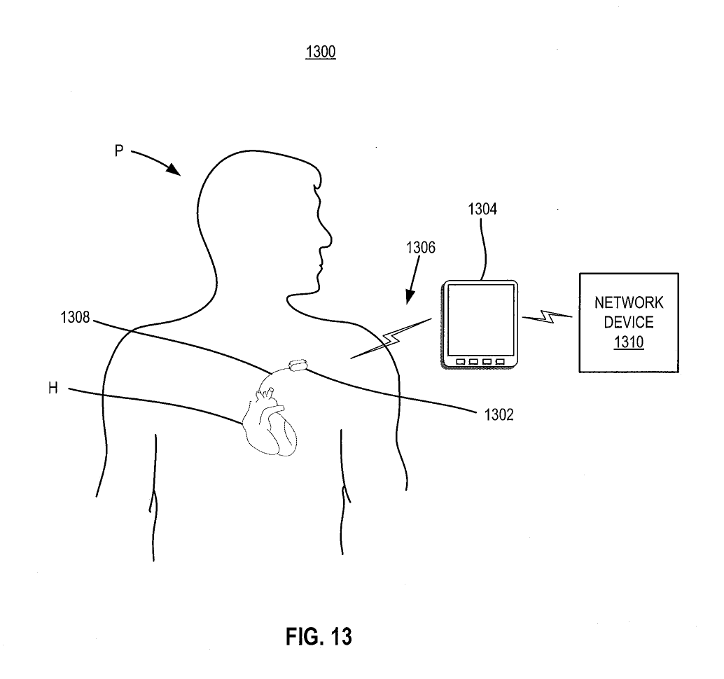

FIG. 13 illustrates a simplified diagram of a device 1302 (implanted within a patient P) that communicates with a device 1304 that is located external to the patient P. The implanted device 1302 and the external device 1304 may communicate with one another via a wireless communication link 1306 (as represented by the depicted wireless symbol).

In the illustrated example, the implanted device 1302 is an implantable cardiac device including one or more leads 1308 that are routed to the heart H of the patient P. One or more of the leads 1308 may include an L-C resonant circuit used for pressure measurements as taught herein. The implanted device 1302 may be a pacemaker, an implantable cardioverter defibrillator, or some other similar device. It should be appreciated, however, that the implanted device 1302 may take other forms.

The external device 1304 also may take various forms. For example, the external device 1304 may be a base station, a programmer, a home safety monitor, a personal monitor, a follow-up monitor, a wearable monitor, or some other type of device that is configured to communicate with the implanted device 1302.

The communication link 1306 may be used to transfer information between the devices 1302 and 1304 in conjunction with various applications such as remote home-monitoring, clinical visits, data acquisition, remote follow-up, and portable or wearable patient monitoring/control systems. For example, information (e.g., pressure information) may be transferred between the devices 1302 and 1304 when the patient P is at a location that is relatively close to the external device 1304. Here, information transfers may be invoked upon command, at designated times, or in some other manner.

As discussed above, an external device may send information it receives from an implanted device to another device (e.g., that may provide a more convenient means for a physician or other personnel to review the information). For example, the external device 1304 may send the information to a network device 1310 (e.g., via a web server). In this way, monitoring personnel (e.g., a physician) may remotely access the information (e.g., by accessing a website). The monitoring personnel may then review the information uploaded from the implantable device to determine whether medical intervention is warranted.

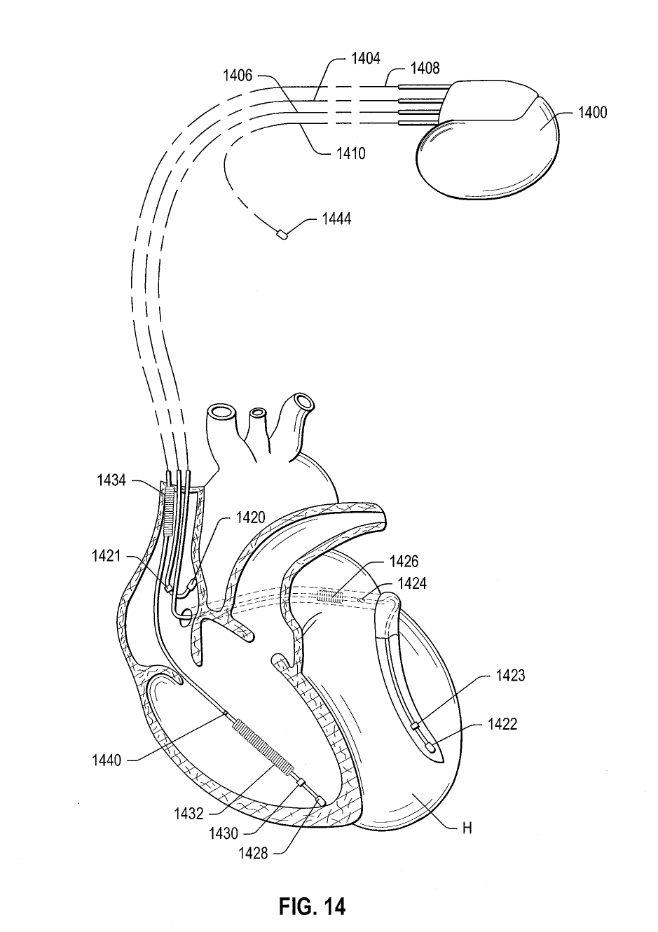

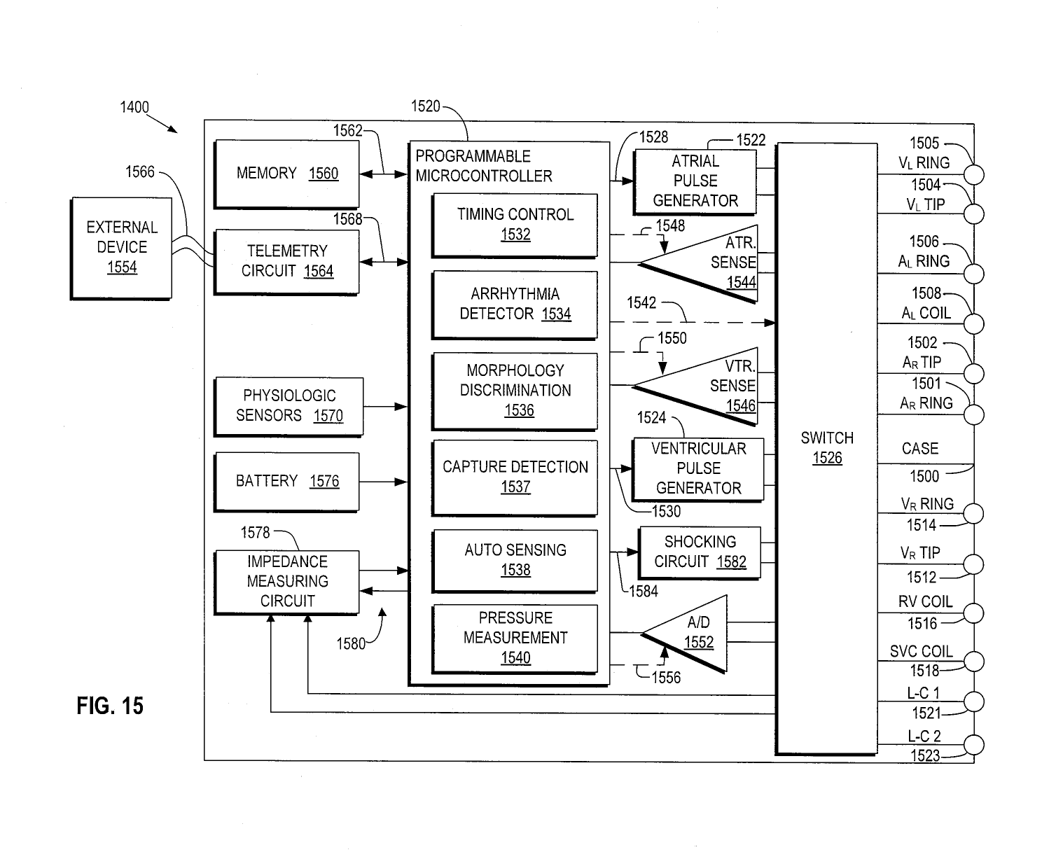

Referring now to FIGS. 14 and 15, an example of an implantable cardiac device 1400 (e.g., a stimulation device such as an implantable cardioverter defibrillator, a pacemaker, etc.) that may be configured to provide pressure monitoring in accordance with the teachings herein will be described. It is to be appreciated and understood that other cardiac devices, including those that are not necessarily implantable, may be used and that the description below is given, in its specific context, to assist the reader in understanding, with more clarity, sample uses of the embodiments described herein.

In various embodiments, the device 1400 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

FIG. 14 shows an exemplary implantable cardiac device 1400 in electrical communication with a patient's heart H by way of three leads 1404, 1406, and 1408, suitable for delivering multi-chamber stimulation and shock therapy. Bodies of the leads 1404, 1406, and 1408 may be formed of silicone, polyurethane, plastic, or similar biocompatible materials to facilitate implant within a patient. Each lead includes one or more conductors, each of which may couple one or more electrodes incorporated into the lead to a connector on the proximal end of the lead. Each connector, in turn, is configured to couple with a complimentary connector (e.g., implemented within a header) of the device 1400.

To sense atrial cardiac signals and to provide right atrial chamber stimulation therapy, the device 1400 is coupled to an implantable right atrial lead 1404 having, for example, an atrial tip electrode 1420, which typically is implanted in the patient's right atrial appendage or septum. FIG. 14 also shows the right atrial lead 1404 as having an optional atrial ring electrode 1421.

To sense left atrial and ventricular cardiac signals and to provide left chamber pacing therapy, the device 1400 is coupled to a coronary sinus lead 1406 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 1406 is designed to receive atrial and ventricular cardiac signals and to deliver left ventricular pacing therapy using, for example, a left ventricular tip electrode 1422 and, optionally, a left ventricular ring electrode 1423; provide left atrial pacing therapy using, for example, a left atrial ring electrode 1424; and provide shocking therapy using, for example, a left atrial coil electrode 1426 (or other electrode capable of delivering a shock). For a more detailed description of a coronary sinus lead, the reader is directed to U.S. Pat. No. 5,466,254, “Coronary Sinus Lead with Atrial Sensing Capability” (Helland), which is incorporated herein by reference.

The device 1400 is also shown in electrical communication with the patient's heart H by way of an implantable right ventricular lead 1408 having, in this implementation, a right ventricular tip electrode 1428, a right ventricular ring electrode 1430, a right ventricular (RV) coil electrode 1432 (or other electrode capable of delivering a shock), and a superior vena cava (SVC) coil electrode 1434 (or other electrode capable of delivering a shock). Typically, the right ventricular lead 1408 is transvenously inserted into the heart H to place the right ventricular tip electrode 1428 in the right ventricular apex so that the RV coil electrode 1432 will be positioned in the right ventricle and the SVC coil electrode 1434 will be positioned in the superior vena cava. Accordingly, the right ventricular lead 1408 is capable of sensing or receiving cardiac signals, and delivering stimulation in the form of pacing and shock therapy to the right ventricle.

The right ventricular lead 1408 also includes an L-C resonant circuit-based pressure sensor component 1440 for monitoring pressure in the right ventricle. It should be appreciated that similar pressure sensor components may be incorporated into other leads and/or at different locations on a given lead.

The device 1400 is also shown in electrical communication with a lead 1410 including one or more components 1444 such as a physiologic sensor. The component 1444 may be positioned in, near or remote from the heart.

It should be appreciated that the device 1400 may connect to leads other than those specifically shown. In addition, the leads connected to the device 1400 may include components other than those specifically shown. For example, a lead may include other types of electrodes, sensors or devices that serve to otherwise interact with a patient or the surroundings.

FIG. 15 depicts an exemplary, simplified block diagram illustrating sample components of the device 1400. A housing 1500 for the device 1400 is often referred to as the “can”, “case” or “case electrode”, and may be programmably selected to act as the return electrode for all “unipolar” modes. The housing 1500 may further be used as a return electrode alone or in combination with one or more of the coil electrodes 1426, 1432 and 1434 for shocking purposes. The housing 1500 may be constructed of a biocompatible material (e.g., titanium) to facilitate implant within a patient.

The housing 1500 further includes a connector (not shown) having a plurality of terminals 1501, 1502, 1504, 1505, 1506, 1508, 1512, 1514, 1516 and 1518 (shown schematically and, for convenience, the names of the electrodes to which they are connected are shown next to the terminals). The connector may be configured to include various other terminals (e.g., terminals 1521 and 1523 coupled to an antenna) depending on the requirements of a given application.

To achieve right atrial sensing and pacing, the connector includes, for example, a right atrial tip terminal (AR TIP) 1502 adapted for connection to the right atrial tip electrode 1420. A right atrial ring terminal (AR RING) 1501 may also be included and adapted for connection to the right atrial ring electrode 1421. To achieve left chamber sensing, pacing, and shocking, the connector includes, for example, a left ventricular tip terminal (VL TIP) 1504, a left ventricular ring terminal (VL RING) 1505, a left atrial ring terminal (AL RING) 1506, and a left atrial shocking terminal (AL COIL) 1508, which are adapted for connection to the left ventricular tip electrode 1422, the left ventricular ring electrode 1423, the left atrial ring electrode 1424, and the left atrial coil electrode 1426, respectively.

To support right chamber sensing, pacing, and shocking, the connector further includes a right ventricular tip terminal (VR TIP) 1512, a right ventricular ring terminal (VR RING) 1514, a right ventricular shocking terminal (RV COIL) 1516, and a superior vena cava shocking terminal (SVC COIL) 1518, which are adapted for connection to the right ventricular tip electrode 1428, the right ventricular ring electrode 1430, the RV coil electrode 1432, and the SVC coil electrode 1434, respectively.

At the core of the device 1400 is a programmable microcontroller 1520 that controls the various modes of stimulation therapy. As is well known in the art, microcontroller 1520 typically includes a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 1520 includes the ability to process or monitor input signals (data or information) as controlled by a program code stored in a designated block of memory. The type of microcontroller is not critical to the described implementations. Rather, any suitable microcontroller 1520 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. Nos. 4,712,555 (Thornander et al.) and 4,944,298 (Sholder), all of which are incorporated by reference herein. For a more detailed description of the various timing intervals that may be used within the device and their inter-relationship, see U.S. Pat. No. 4,788,980 (Mann et al.), also incorporated herein by reference.

FIG. 15 also shows an atrial pulse generator 1522 and a ventricular pulse generator 1524 that generate pacing stimulation pulses for delivery by the right atrial lead 1404, the coronary sinus lead 1406, the right ventricular lead 1408, or some combination of these leads via an electrode configuration switch 1526. It is understood that in order to provide stimulation therapy in each of the four chambers of the heart, the atrial and ventricular pulse generators 1522 and 1524 may include dedicated, independent pulse generators, multiplexed pulse generators, or shared pulse generators. The pulse generators 1522 and 1524 are controlled by the microcontroller 1520 via appropriate control signals 1528 and 1530, respectively, to trigger or inhibit the stimulation pulses.

Microcontroller 1520 further includes timing control circuitry 1532 to control the timing of the stimulation pulses (e.g., pacing rate, atrio-ventricular (A-V) delay, atrial interconduction (A-A) delay, or ventricular interconduction (V-V) delay, etc.) or other operations, as well as to keep track of the timing of refractory periods, blanking intervals, noise detection windows, evoked response windows, alert intervals, marker channel timing, etc., as known in the art.

Microcontroller 1520 further includes an arrhythmia detector 1534. The arrhythmia detector 1534 may be utilized by the device 1400 for determining desirable times to administer various therapies. The arrhythmia detector 1534 may be implemented, for example, in hardware as part of the microcontroller 1520, or as software/firmware instructions programmed into the device 1400 and executed on the microcontroller 1520 during certain modes of operation.

Microcontroller 1520 may include a morphology discrimination module 1536, a capture detection module 1537 and an auto sensing module 1538. These modules are optionally used to implement various exemplary recognition algorithms or methods. The aforementioned components may be implemented, for example, in hardware as part of the microcontroller 1520, or as software/firmware instructions programmed into the device 1400 and executed on the microcontroller 1520 during certain modes of operation.

The configuration switch 1526 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 1526, in response to a control signal 1542 from the microcontroller 1520, may be used to determine the polarity of the stimulation pulses (e.g., unipolar, bipolar, combipolar, etc.) by selectively closing the appropriate combination of switches (not shown) as is known in the art.

Atrial sensing circuits (ATR. SENSE) 1544 and ventricular sensing circuits (VTR. SENSE) 1546 may also be selectively coupled to the right atrial lead 1404, coronary sinus lead 1406, and the right ventricular lead 1408, through the switch 1526 for detecting the presence of cardiac activity in each of the four chambers of the heart. Accordingly, the atrial and ventricular sensing circuits 1544 and 1546 may include dedicated sense amplifiers, multiplexed amplifiers, or shared amplifiers. Switch 1526 determines the “sensing polarity” of the cardiac signal by selectively closing the appropriate switches, as is also known in the art. In this way, the clinician may program the sensing polarity independent of the stimulation polarity. The sensing circuits (e.g., circuits 1544 and 1546) are optionally capable of obtaining information indicative of tissue capture.

Each sensing circuit 1544 and 1546 preferably employs one or more low power, precision amplifiers with programmable gain, automatic gain control, bandpass filtering, a threshold detection circuit, or some combination of these components, to selectively sense the cardiac signal of interest. The automatic gain control enables the device 1400 to deal effectively with the difficult problem of sensing the low amplitude signal characteristics of atrial or ventricular fibrillation.

The outputs of the atrial and ventricular sensing circuits 1544 and 1546 are connected to the microcontroller 1520, which, in turn, is able to trigger or inhibit the atrial and ventricular pulse generators 1522 and 1524, respectively, in a demand fashion in response to the absence or presence of cardiac activity in the appropriate chambers of the heart. Furthermore, as described herein, the microcontroller 1520 is also capable of analyzing information output from the sensing circuits 1544 and 1546, a data acquisition system 1552, or both. This information may be used to determine or detect whether and to what degree tissue capture has occurred and to program a pulse, or pulses, in response to such determinations. The sensing circuits 1544 and 1546, in turn, receive control signals over signal lines 1548 and 1550, respectively, from the microcontroller 1520 for purposes of controlling the gain, threshold, polarization charge removal circuitry (not shown), and the timing of any blocking circuitry (not shown) coupled to the inputs of the sensing circuits 1544 and 1546 as is known in the art.

For arrhythmia detection, the device 1400 utilizes the atrial and ventricular sensing circuits 1544 and 1546 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 1534 of the microcontroller 1520 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (A/D) data acquisition system 1552. The data acquisition system 1552 is configured (e.g., via signal line 1556) to acquire intracardiac electrogram (“IEGM”) signals or other signals, convert the raw analog data into a digital signal, and store the digital signals for later processing, for telemetric transmission to an external device 1554, or both. For example, the data acquisition system 1552 may be coupled to the right atrial lead 1404, the coronary sinus lead 1406, the right ventricular lead 1408 and other leads through the switch 1526 to sample cardiac signals across any pair of desired electrodes.

The data acquisition system 1552 also may be coupled to receive signals from other input devices. For example, the data acquisition system 1552 may sample signals from a physiologic sensor 1570 or other components shown in FIG. 15 (connections not shown).

The microcontroller 1520 is further coupled to a memory 1560 by a suitable data/address bus 1562, wherein the programmable operating parameters used by the microcontroller 1520 are stored and modified, as required, in order to customize the operation of the device 1400 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 1552), which data may then be used for subsequent analysis to guide the programming of the device 1400.

Advantageously, the operating parameters of the implantable device 1400 may be non-invasively programmed into the memory 1560 through a telemetry circuit 1564 in telemetric communication via communication link 1566 with the external device 1554, such as a programmer, transtelephonic transceiver, a diagnostic system analyzer or some other device. The microcontroller 1520 activates the telemetry circuit 1564 with a control signal (e.g., via bus 1568). The telemetry circuit 1564 advantageously allows intracardiac electrograms and status information relating to the operation of the device 1400 (as contained in the microcontroller 1520 or memory 1560) to be sent to the external device 1554 through an established communication link 1566.

The device 1400 can further include one or more physiologic sensors 1570. In some embodiments, the device 1400 may include a “rate-responsive” sensor that may provide, for example, information to aid in adjustment of pacing stimulation rate according to the exercise state of the patient. One or more physiologic sensors 1570 (e.g., a pressure sensor) may further be used to detect changes in cardiac output, changes in the physiological condition of the heart, or diurnal changes in activity (e.g., detecting sleep and wake states). Accordingly, the microcontroller 1520 responds by adjusting the various pacing parameters (such as rate, A-V Delay, V-V Delay, etc.) at which the atrial and ventricular pulse generators 1522 and 1524 generate stimulation pulses.

While shown as being included within the device 1400, it is to be understood that a physiologic sensor 1570 may also be external to the device 1400, yet still be implanted within or carried by the patient. Examples of physiologic sensors that may be implemented in conjunction with the device 1400 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), which patent is hereby incorporated by reference.

The one or more physiologic sensors 1570 may optionally include one or more of components to help detect movement (via, e.g., a position sensor or an accelerometer) and minute ventilation (via an MV sensor) in the patient. Signals generated by the position sensor and MV sensor may be passed to the microcontroller 1520 for analysis in determining whether to adjust the pacing rate, etc. The microcontroller 1520 may thus monitor the signals for indications of the patient's position and activity status, such as whether the patient is climbing up stairs or descending down stairs or whether the patient is sitting up after lying down.

The device 1400 additionally includes a battery 1576 that provides operating power to all of the circuits shown in FIG. 15. For a device 1400 which employs shocking therapy, the battery 1576 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 1576 also desirably has a predictable discharge characteristic so that elective replacement time can be detected. Accordingly, the device 1400 preferably employs lithium or other suitable battery technology.

The device 1400 can further include magnet detection circuitry (not shown), coupled to the microcontroller 1520, to detect when a magnet is placed over the device 1400. A magnet may be used by a clinician to perform various test functions of the device 1400 and to signal the microcontroller 1520 that the external device 1554 is in place to receive data from or transmit data to the microcontroller 1520 through the telemetry circuit 1564.

The device 1400 further includes an impedance measuring circuit 1578 that is enabled by the microcontroller 1520 via a control signal 1580. The known uses for an impedance measuring circuit 1578 include, but are not limited to, lead impedance surveillance during the acute and chronic phases for proper performance, lead positioning or dislodgement; detecting operable electrodes and automatically switching to an operable pair if dislodgement occurs; measuring respiration or minute ventilation; measuring thoracic impedance for determining shock thresholds; detecting when the device 1400 has been implanted; measuring stroke volume; and detecting the opening of heart valves, etc. The impedance measuring circuit 1578 is advantageously coupled to the switch 1526 so that any desired electrode may be used.

In the case where the device 1400 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 1520 further controls a shocking circuit 1582 by way of a control signal 1584. The shocking circuit 1582 generates shocking pulses of low (e.g., up to 0.5 J), moderate (e.g., 0.5 J to 10 J), or high energy (e.g., 11 J to 40 J), as controlled by the microcontroller 1520. Such shocking pulses are applied to the patient's heart H through, for example, two shocking electrodes and as shown in this embodiment, selected from the left atrial coil electrode 1426, the RV coil electrode 1432 and the SVC coil electrode 1434. As noted above, the housing 1500 may act as an active electrode in combination with the RV coil electrode 1432, as part of a split electrical vector using the SVC coil electrode 1434 or the left atrial coil electrode 1426 (i.e., using the RV electrode as a common electrode), or in some other arrangement.

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining to the treatment of fibrillation. Accordingly, the microcontroller 1520 is capable of controlling the synchronous or asynchronous delivery of the shocking pulses.

As mentioned above, the device 1400 may include several components that support pressure measurement functionality as taught herein. For example, one or more of the switch 1526, the sense circuits 1544, 1546, and the data acquisition system 1552 may be used to receive oscillating signals from an L-C resonant circuit. The data described above may be stored in the memory 1560.

The microcontroller 1520 (e.g., a processor providing signal processing functionality) also may implement or support at least a portion of the pressure measurement functionality 1540 discussed herein. For example, the microcontroller 1520 may include a pressure measurement component 1540 (e.g., implemented in hardware or in hardware and executable code) operable to control the coupling of an excitation signal to an L-C resonant circuit and to process data based on a received oscillating signal.

It should be appreciated from the above that the various structures and functions described herein may be incorporated into a variety of apparatuses (e.g., a pacing device, a monitoring device, etc.) and implemented in a variety of ways. Different embodiments of such an apparatus may include a variety of hardware and software processing components. In some embodiments, hardware components such as processors, controllers, state machines, logic, or some combination of these components, may be used to implement some of the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

The components and functions described herein may be connected or coupled in many different ways. The manner in which this is done may depend, in part, on whether and how the components are separated from the other components. In some cases, components may be directly coupled (i.e., without intervening component other than connections), while in other cases components may be indirectly coupled (i.e., via one or more intervening components). In some embodiments, some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in any methods (e.g., processes) disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements. In addition, terminology of the form “at least one of A, B, or C” or “one or more of A, B, or C” or “at least one of the group consisting of A, B, and C” used in the description or the claims means “A or B or C or any combination of these elements.”

As used herein, the term “determining” encompasses a wide variety of actions. For example, “determining” may include calculating, computing, processing, deriving, investigating, looking up (e.g., looking up in a table, a database or another data structure), ascertaining, and the like. Also, “determining” may include receiving (e.g., receiving information), accessing (e.g., accessing data in a memory), and the like. Also, “determining” may include resolving, selecting, choosing, establishing, and the like.

In some aspects, an apparatus or any component of an apparatus may be configured to (or operable to or adapted to) provide functionality as taught herein. This may be achieved, for example: by manufacturing (e.g., fabricating) the apparatus or component so that it will provide the functionality; by programming the apparatus or component so that it will provide the functionality; or through the use of some other suitable implementation technique.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above, it will be understood that the teachings herein are intended to cover any changes, adaptations or modifications that are within the scope of the disclosure.

What is claimed is:

1. An implantable medical device for measuring pressure via an inductor-capacitor resonant circuit of an implantable lead, comprising:
an antenna;
a connector comprising at least one first connector terminal, and operable to interface with at least one lead terminal of the implantable lead to couple the inductor-capacitor resonant circuit to the at least one connector terminal;
a switch comprising at least one first switch terminal coupled to the antenna, at least one second switch terminal coupled to the at least one connector terminal, and at least one control terminal, wherein the switch is operable to selectively couple the at least one first switch terminal to the at least one second switch terminal based on a control signal received via the at least one control terminal; and
a control circuit coupled to the antenna, the at least one control terminal and the at least one connector terminal, and operable to generate the control signal based on whether a radio frequency signal having a frequency substantially equal to a nominal resonant frequency of the inductor-capacitor resonant circuit is received via the antenna, and further operable to process signals received from the inductor-capacitor resonant circuit via the at least one connector terminal to generate data representative of pressure induced on the implantable lead.

2. The device of claim 1, wherein:
the control circuit comprises a radio frequency transceiver operable to receive a message via the antenna from an external device;
the control circuit is further operable to determine whether the radio frequency signal is received; and
the determination of whether the radio frequency signal is received comprises decoding the message to determine whether the message indicates that the external device will commence transmission of the radio frequency signal.

3. The device of claim 1, wherein:
the control circuit comprises a radio frequency transceiver operable to receive a message via the antenna from an external device;
the control circuit is further operable to determine whether the radio frequency signal is received; and
the determination of whether the radio frequency signal is received comprises decoding the message to determine whether the external device has sent a request to close the switch or open the switch.

4. The device of claim 1, wherein the control circuit is further operable to generate the control signal to close the switch for a defined period of time.

5. The device of claim 1, wherein:
the control circuit comprises a radio frequency detector circuit operable to detect the radio frequency signal having a frequency substantially equal to a nominal resonant frequency of the inductor-capacitor resonant circuit; and
the control circuit is further operable to generate the control signal based on whether the detector circuit detects the radio frequency signal.

6. The device of claim 1, wherein the control circuit comprises a radio frequency transceiver operable to communicate via the antenna with an external device to send the generated data to the external device.

7. The device of claim 1, wherein the processing of the signals received from the inductor-capacitor resonant circuit to generate the data comprises:
processing the received signals to determine at least one frequency of the signals; and
determining at least one pressure value based on the determined at least one frequency, wherein the generated data comprises the determined at least one pressure value.

8. The device of claim 1, wherein:
the device further comprises a housing defining a first interior space;
the control circuit is located within the first interior space defined by the housing;
the device further comprises a header attached to the housing and defining a second interior space; and
the antenna is located at least partially within the second interior space defined by the header.

9. An implantable medical system, comprising:
an implantable lead comprising:
a biocompatible lead body;
an inductor-capacitor resonant circuit located within the lead body, and comprising an inductive circuit and a flexible capacitive circuit electrically coupled in parallel;
at least one lead terminal located at a proximal section of the lead body, and electrically coupled to the inductor-capacitor resonant circuit; and
a flexible insulator material located adjacent an exterior surface of the lead body and engaged with the capacitive circuit to couple pressure waves to the capacitive circuit; and
an implantable medical device comprising:
an antenna;
a second connector comprising at least one connector terminal, and operable to interface with the implantable lead to couple the at least one connector terminal to the at least one lead terminal;
a switch comprising at least one first switch terminal electrically coupled to the antenna, at least one second switch terminal electrically coupled to the at least one connector terminal, and at least one control terminal, wherein the switch is operable to selectively couple the at least one first switch terminal to the at least one second switch terminal based on a control signal received via the at least one control terminal; and
a control circuit coupled to the antenna, the at least one control terminal and the at least one connector terminal, and operable to generate the control signal based on whether a radio frequency signal having a frequency substantially equal to a nominal resonant frequency of the inductor-capacitor resonant circuit is received via the antenna, and further operable to process signals received from the inductor-capacitor resonant circuit via the at least one connector terminal to generate data representative of pressure induced on the implantable lead.

10. The system of claim 9, wherein the inductor-capacitor resonant circuit is not electrically coupled to any other electrical component of the implantable lead other than the at least one lead terminal.

11. An implantable medical device for measuring pressure via an inductor-capacitor resonant circuit of an implantable lead, comprising:
a connector comprising at least one connector terminal, and operable to interface with at least one lead terminal of the implantable lead to couple the inductor-capacitor resonant circuit to the at least one connector terminal;
a signal generator comprising at least one output terminal coupled to the at least one connector terminal, and operable to output an excitation signal having a frequency substantially equal to a nominal resonant frequency of the inductor-capacitor resonant circuit at the at least one output terminal, wherein the signal generator is further operable to output the excitation signal based on a control signal received via at least one control terminal; and
a control circuit coupled to the at least one control terminal and the at least one connector terminal, and operable to generate the control signal to selectively control whether the excitation signal is provided to the inductor-capacitor resonant circuit, and further operable to process signals received from the inductor-capacitor resonant circuit via the at least one connector terminal to generate data representative of pressure induced on the implantable lead.

12. The device of claim 11, wherein the excitation signal comprises at least one pulse signal.

13. The device of claim 11, wherein:
the device further comprises an antenna;
the control circuit comprises a radio frequency transceiver operable to receive a message via the antenna from an external device;
the control circuit is further operable to decode the message to determine whether the external device has sent a request to conduct a pressure measurement; and
the control circuit is further operable to generate the control signal based on the determination of whether the message indicates that the external device has sent a request.

14. The device of claim 11, wherein the control circuit is further operable to generate the control signal to excite the inductor-capacitor resonant circuit for a defined period of time.

15. The device of claim 11, wherein:
the device further comprises an antenna; and
the control circuit comprises a radio frequency transceiver operable to communicate via the antenna with an external device to send the generated data to the external device.

16. The device of claim 11, wherein the processing of the signals received from the inductor-capacitor resonant circuit to generate the data comprises:
processing the received signals to determine at least one frequency of the signals; and
determining at least one pressure value based on the determined at least one frequency, wherein the generated data comprises the determined at least one pressure value.

17. A method of sensing pressure via an inductor-capacitor resonant circuit of an implantable lead, comprising:
determining whether a radio frequency signal having a frequency substantially equal to a nominal resonant frequency of the inductor-capacitor resonant circuit is received via an antenna;
selectively coupling the antenna to the inductor-capacitor resonant circuit based on the determination; and
processing signals received from the inductor-capacitor resonant circuit to generate data representative of pressure induced on the implantable lead.

18. The method of claim 17, further comprising receiving a message via the antenna from an external device, wherein the determination of whether the radio frequency signal is received comprises decoding the message to determine whether the message indicates that the external device will commence transmission of the radio frequency signal.

19. The method of claim 17, further comprising receiving a message via the antenna from an external device, wherein the determination of whether the radio frequency signal is received comprises decoding the message to determine whether the external device has sent a request to close a switch or open the switch.

20. The method of claim 17, further comprising communicating via the antenna with an external device to send the generated data to the external device.

21. The method of claim 17, wherein the processing of the signals received from the inductor-capacitor resonant circuit to generate the data comprises:
processing the received signals to determine at least one frequency of the signals; and
determining at least one pressure value based on the determined at least one frequency, wherein the generated data comprises the determined at least one pressure value.
